SEQUENCE LISTING (13 sequences)

SEQ ID NO: 1 - DNA, 102 bp, Artificial sequence
  Synthetic oligonucleotide
agggggttagttttgcgtattggggtcattggtgttcttgtagttgaaatacaacgatggtttttcatatcattggtcgtggttgtagtccgtgcgagaata

SEQ ID NO: 2 - DNA, 16569 bp, Homo sapiens
  misc_feature (3107)..(3107): n is a, c, g, or t
gatcacaggtctatcaccctattaaccactcacgggagctctccatgcatttggtattttcgtctggggggtatgcacgcgatagcattgcgagacgctggagccggagcaccctatgtcgcagtatctgtctttgattcctgcctcatcctattatttatcgcacctacgttcaatattacaggcgaacatacttactaaagtgtgttaattaattaatgcttgtaggacataataataacaattgaatgtctgcacagccactttccacacagacatcataacaaaaaatttccaccaaaccccccctcccccgcttctggccacagcacttaaacacatctctgccaaaccccaaaaacaaagaaccctaacaccagcctaaccagatttcaaattttatcttttggcggtatgcacttttaacagtcaccccccaactaacacattattttcccctcccactcccatactactaatctcatcaatacaacccccgcccatcctacccagcacacacacaccgctgctaaccccataccccgaaccaaccaaaccccaaagacaccccccacagtttatgtagcttacctcctcaaagcaatacactgaaaatgtttagacgggctcacatcaccccataaacaaataggtttggtcctagcctttctattagctcttagtaagattacacatgcaagcatccccgttccagtgagttcaccctctaaatcaccacgatcaaaaggaacaagcatcaagcacgcagcaatgcagctcaaaacgcttagcctagccacacccccacgggaaacagcagtgattaacctttagcaataaacgaaagtttaactaagctatactaaccccagggttggtcaatttcgtgccagccaccgcggtcacacgattaacccaagtcaatagaagccggcgtaaagagtgttttagatcaccccctccccaataaagctaaaactcacctgagttgtaaaaaactccagttgacacaaaatagactacgaaagtggctttaacatatctgaacacacaatagctaagacccaaactgggattagataccccactatgcttagccctaaacctcaacagttaaatcaacaaaactgctcgccagaacactacgagccacagcttaaaactcaaaggacctggcggtgcttcatatccctctagaggagcctgttctgtaatcgataaaccccgatcaacctcaccacctcttgctcagcctatataccgccatcttcagcaaaccctgatgaaggctacaaagtaagcgcaagtacccacgtaaagacgttaggtcaaggtgtagcccatgaggtggcaagaaatgggctacattttctaccccagaaaactacgatagcccttatgaaacttaagggtcgaaggtggatttagcagtaaactaagagtagagtgcttagttgaacagggccctgaagcgcgtacacaccgcccgtcaccctcctcaagtatacttcaaaggacatttaactaaaacccctacgcatttatatagaggagacaagtcgtaacatggtaagtgtactggaaagtgcacttggacgaaccagagtgtagcttaacacaaagcacccaacttacacttaggagatttcaacttaacttgaccgctctgagctaaacctagccccaaacccactccaccttactaccagacaaccttagccaaaccatttacccaaataaagtataggcgatagaaattgaaacctggcgcaatagatatagtaccgcaagggaaagatgaaaaattataaccaagcataatatagcaaggactaacccctataccttctgcataatgaattaactagaaataactttgcaaggagagccaaagctaagacccccgaaaccagacgagctacctaagaacagctaaaagagcacacccgtctatgtagcaaaatagtgggaagatttataggtagaggcgacaaacctaccgagcctggtgatagctggttgtccaagatagaatcttagttcaactttaaatttgcccacagaaccctctaaatccccttgtaaatttaactgttagtccaaagaggaacagctctttggacactaggaaaaaaccttgtagagagagtaaaaaatttaacacccatagtaggcctaaaagcagccaccaattaagaaagcgttcaagctcaacacccactacctaaaaaatcccaaacatataactgaactcctcacacccaattggaccaatctatcaccctatagaagaactaatgttagtataagtaacatgaaaacattctcctccgcataagcctgcgtcagattaaaacactgaactgacaattaacagcccaatatctacaatcaaccaacaagtcattattaccctcactgtcaacccaacacaggcatgctcataaggaaaggttaaaaaaagtaaaaggaactcggcaaatcttaccccgcctgtttaccaaaaacatcacctctagcatcaccagtattagaggcaccgcctgcccagtgacacatgtttaacggccgcggtaccctaaccgtgcaaaggtagcataatcacttgttccttaaatagggacctgtatgaatggctccacgagggttcagctgtctcttacttttaaccagtgaaattgacctgcccgtgaagaggcgggcataacacagcaagacgagaagaccctatggagctttaatttattaatgcaaacagtacctaacaaacccacaggtcctaaactaccaaacctgcattaaaaatttcggttggggcgacctcggagcagaacccaacctccgagcagtacatgctaagacttcaccagtcaaagcgaactactatactcaattgatccaataacttgaccaacggaacaagttaccctagggataacagcgcaatcctattctagagtccatatcaacaatagggtttacgacctcgatgttggatcaggacatcccgatggtgcagccgctattaaaggttcgtttgttcaacgattaaagtcctacgtgatctgagttcagaccggagtaatccaggtcggtttctatctacnttcaaattcctccctgtacgaaaggacaagagaaataaggcctacttcacaaagcgccttcccccgtaaatgatatcatctcaacttagtattatacccacacccacccaagaacagggtttgttaagatggcagagcccggtaatcgcataaaacttaaaactttacagtcagaggttcaattcctcttcttaacaacatacccatggccaacctcctactcctcattgtacccattctaatcgcaatggcattcctaatgcttaccgaacgaaaaattctaggctatatacaactacgcaaaggccccaacgttgtaggcccctacgggctactacaacccttcgctgacgccataaaactcttcaccaaagagcccctaaaacccgccacatctaccatcaccctctacatcaccgccccgaccttagctctcaccatcgctcttctactatgaacccccctccccatacccaaccccctggtcaacctcaacctaggcctcctatttattctagccacctctagcctagccgtttactcaatcctctgatcagggtgagcatcaaactcaaactacgccctgatcggcgcactgcgagcagtagcccaaacaatctcatatgaagtcaccctagccatcattctactatcaacattactaataagtggctcctttaacctctccacccttatcacaacacaagaacacctctgattactcctgccatcatgacccttggccataatatgatttatctccacactagcagagaccaaccgaacccccttcgaccttgccgaaggggagtccgaactagtctcaggcttcaacatcgaatacgccgcaggccccttcgccctattcttcatagccgaatacacaaacattattataataaacaccctcaccactacaatcttcctaggaacaacatatgacgcactctcccctgaactctacacaacatattttgtcaccaagaccctacttctaacctccctgttcttatgaattcgaacagcatacccccgattccgctacgaccaactcatacacctcctatgaaaaaacttcctaccactcaccctagcattacttatatgatatgtctccatacccattacaatctccagcattccccctcaaacctaagaaatatgtctgataaaagagttactttgatagagtaaataataggagcttaaacccccttatttctaggactatgagaatcgaacccatccctgagaatccaaaattctccgtgccacctatcacaccccatcctaaagtaaggtcagctaaataagctatcgggcccataccccgaaaatgttggttatacccttcccgtactaattaatcccctggcccaacccgtcatctactctaccatctttgcaggcacactcatcacagcgctaagctcgcactgattttttacctgagtaggcctagaaataaacatgctagcttttattccagttctaaccaaaaaaataaaccctcgttccacagaagctgccatcaagtatttcctcacgcaagcaaccgcatccataatccttctaatagctatcctcttcaacaatatactctccggacaatgaaccataaccaatactaccaatcaatactcatcattaataatcataatagctatagcaataaaactaggaatagccccctttcacttctgagtcccagaggttacccaaggcacccctctgacatccggcctgcttcttctcacatgacaaaaactagcccccatctcaatcatataccaaatctctccctcactaaacgtaagccttctcctcactctctcaatcttatccatcatagcaggcagttgaggtggattaaaccaaacccagctacgcaaaatcttagcatactcctcaattacccacataggatgaataatagcagttctaccgtacaaccctaacataaccattcttaatttaactatttatattatcctaactactaccgcattcctactactcaacttaaactccagcaccacgaccctactactatctcgcacctgaaacaagctaacatgactaacacccttaattccatccaccctcctctccctaggaggcctgcccccgctaaccggctttttgcccaaatgggccattatcgaagaattcacaaaaaacaatagcctcatcatccccaccatcatagccaccatcaccctccttaacctctacttctacctacgcctaatctactccacctcaatcacactactccccatatctaacaacgtaaaaataaaatgacagtttgaacatacaaaacccaccccattcctccccacactcatcgcccttaccacgctactcctacctatctccccttttatactaataatcttatagaaatttaggttaaatacagaccaagagccttcaaagccctcagtaagttgcaatacttaatttctgtaacagctaaggactgcaaaaccccactctgcatcaactgaacgcaaatcagccactttaattaagctaagcccttactagaccaatgggacttaaacccacaaacacttagttaacagctaagcaccctaatcaactggcttcaatctacttctcccgccgccgggaaaaaaggcgggagaagccccggcaggtttgaagctgcttcttcgaatttgcaattcaatatgaaaatcacctcggagctggtaaaaagaggcctaacccctgtctttagatttacagtccaatgcttcactcagccattttacctcacccccactgatgttcgccgaccgttgactattctctacaaaccacaaagacattggaacactatacctattattcggcgcatgagctggagtcctaggcacagctctaagcctccttattcgagccgagctgggccagccaggcaaccttctaggtaacgaccacatctacaacgttatcgtcacagcccatgcatttgtaataatcttcttcatagtaatacccatcataatcggaggctttggcaactgactagttcccctaataatcggtgcccccgatatggcgtttccccgcataaacaacataagcttctgactcttacctccctctctcctactcctgctcgcatctgctatagtggaggccggagcaggaacaggttgaacagtctaccctcccttagcagggaactactcccaccctggagcctccgtagacctaaccatcttctccttacacctagcaggtgtctcctctatcttaggggccatcaatttcatcacaacaattatcaatataaaaccccctgccataacccaataccaaacgcccctcttcgtctgatccgtcctaatcacagcagtcctacttctcctatctctcccagtcctagctgctggcatcactatactactaacagaccgcaacctcaacaccaccttcttcgaccccgccggaggaggagaccccattctataccaacacctattctgatttttcggtcaccctgaagtttatattcttatcctaccaggcttcggaataatctcccatattgtaacttactactccggaaaaaaagaaccatttggatacataggtatggtctgagctatgatatcaattggcttcctagggtttatcgtgtgagcacaccatatatttacagtaggaatagacgtagacacacgagcatatttcacctccgctaccataatcatcgctatccccaccggcgtcaaagtatttagctgactcgccacactccacggaagcaatatgaaatgatctgctgcagtgctctgagccctaggattcatctttcttttcaccgtaggtggcctgactggcattgtattagcaaactcatcactagacatcgtactacacgacacgtactacgttgtagcccacttccactatgtcctatcaataggagctgtatttgccatcataggaggcttcattcactgatttcccctattctcaggctacaccctagaccaaacctacgccaaaatccatttcactatcatattcatcggcgtaaatctaactttcttcccacaacactttctcggcctatccggaatgccccgacgttactcggactaccccgatgcatacaccacatgaaacatcctatcatctgtaggctcattcatttctctaacagcagtaatattaataattttcatgatttgagaagccttcgcttcgaagcgaaaagtcctaatagtagaagaaccctccataaacctggagtgactatatggatgccccccaccctaccacacattcgaagaacccgtatacataaaatctagacaaaaaaggaaggaatcgaaccccccaaagctggtttcaagccaaccccatggcctccatgactttttcaaaaaggtattagaaaaaccatttcataactttgtcaaagttaaattataggctaaatcctatatatcttaatggcacatgcagcgcaagtaggtctacaagacgctacttcccctatcatagaagagcttatcacctttcatgatcacgccctcataatcattttccttatctgcttcctagtcctgtatgcccttttcctaacactcacaacaaaactaactaatactaacatctcagacgctcaggaaatagaaaccgtctgaactatcctgcccgccatcatcctagtcctcatcgccctcccatccctacgcatcctttacataacagacgaggtcaacgatccctcccttaccatcaaatcaattggccaccaatggtactgaacctacgagtacaccgactacggcggactaatcttcaactcctacatacttcccccattattcctagaaccaggcgacctgcgactccttgacgttgacaatcgagtagtactcccgattgaagcccccattcgtataataattacatcacaagacgtcttgcactcatgagctgtccccacattaggcttaaaaacagatgcaattcccggacgtctaaaccaaaccactttcaccgctacacgaccgggggtatactacggtcaatgctctgaaatctgtggagcaaaccacagtttcatgcccatcgtcctagaattaattcccctaaaaatctttgaaatagggcccgtatttaccctatagcaccccctctaccccctctagagcccactgtaaagctaacttagcattaaccttttaagttaaagattaagagaaccaacacctctttacagtgaaatgccccaactaaatactaccgtatggcccaccataattacccccatactccttacactattcctcatcacccaactaaaaatattaaacacaaactaccacctacctccctcaccaaagcccataaaaataaaaaattataacaaaccctgagaaccaaaatgaacgaaaatctgttcgcttcattcattgcccccacaatcctaggcctacccgccgcagtactgatcattctatttccccctctattgatccccacctccaaatatctcatcaacaaccgactaatcaccacccaacaatgactaatcaaactaacctcaaaacaaatgataaccatacacaacactaaaggacgaacctgatctcttatactagtatccttaatcatttttattgccacaactaacctcctcggactcctgcctcactcatttacaccaaccacccaactatctataaacctagccatggccatccccttatgagcgggcacagtgattataggctttcgctctaagattaaaaatgccctagcccacttcttaccacaaggcacacctacaccccttatccccatactagttattatcgaaaccatcagcctactcattcaaccaatagccctggccgtacgcctaaccgctaacattactgcaggccacctactcatgcacctaattggaagcgccaccctagcaatatcaaccattaaccttccctctacacttatcatcttcacaattctaattctactgactatcctagaaatcgctgtcgccttaatccaagcctacgttttcacacttctagtaagcctctacctgcacgacaacacataatgacccaccaatcacatgcctatcatatagtaaaacccagcccatgacccctaacaggggccctctcagccctcctaatgacctccggcctagccatgtgatttcacttccactccataacgctcctcatactaggcctactaaccaacacactaaccatataccaatgatggcgcgatgtaacacgagaaagcacataccaaggccaccacacaccacctgtccaaaaaggccttcgatacgggataatcctatttattacctcagaagtttttttcttcgcaggatttttctgagccttttaccactccagcctagcccctaccccccaattaggagggcactggcccccaacaggcatcaccccgctaaatcccctagaagtcccactcctaaacacatccgtattactcgcatcaggagtatcaatcacctgagctcaccatagtctaatagaaaacaaccgaaaccaaataattcaagcactgcttattacaattttactgggtctctattttaccctcctacaagcctcagagtacttcgagtctcccttcaccatttccgacggcatctacggctcaacattttttgtagccacaggcttccacggacttcacgtcattattggctcaactttcctcactatctgcttcatccgccaactaatatttcactttacatccaaacatcactttggcttcgaagccgccgcctgatactggcattttgtagatgtggtttgactatttctgtatgtctccatctattgatgagggtcttactcttttagtataaatagtaccgttaacttccaattaactagttttgacaacattcaaaaaagagtaataaacttcgccttaattttaataatcaacaccctcctagccttactactaataattattacattttgactaccacaactcaacggctacatagaaaaatccaccccttacgagtgcggcttcgaccctatatcccccgcccgcgtccctttctccataaaattcttcttagtagctattaccttcttattatttgatctagaaattgccctccttttacccctaccatgagccctacaaacaactaacctgccactaatagttatgtcatccctcttattaatcatcatcctagccctaagtctggcctatgagtgactacaaaaaggattagactgaaccgaattggtatatagtttaaacaaaacgaatgatttcgactcattaaattatgataatcatatttaccaaatgcccctcatttacataaatattatactagcatttaccatctcacttctaggaatactagtatatcgctcacacctcatatcctccctactatgcctagaaggaataatactatcgctgttcattatagctactctcataaccctcaacacccactccctcttagccaatattgtgcctattgccatactagtctttgccgcctgcgaagcagcggtgggcctagccctactagtctcaatctccaacacatatggcctagactacgtacataacctaaacctactccaatgctaaaactaatcgtcccaacaattatattactaccactgacatgactttccaaaaaacacataatttgaatcaacacaaccacccacagcctaattattagcatcatccctctactattttttaaccaaatcaacaacaacctatttagctgttccccaaccttttcctccgaccccctaacaacccccctcctaatactaactacctgactcctacccctcacaatcatggcaagccaacgccacttatccagtgaaccactatcacgaaaaaaactctacctctctatactaatctccctacaaatctccttaattataacattcacagccacagaactaatcatattttatatcttcttcgaaaccacacttatccccaccttggctatcatcacccgatgaggcaaccagccagaacgcctgaacgcaggcacatacttcctattctacaccctagtaggctcccttcccctactcatcgcactaatttacactcacaacaccctaggctcactaaacattctactactcactctcactgcccaagaactatcaaactcctgagccaacaacttaatatgactagcttacacaatagcttttatagtaaagatacctctttacggactccacttatgactccctaaagcccatgtcgaagcccccatcgctgggtcaatagtacttgccgcagtactcttaaaactaggcggctatggtataatacgcctcacactcattctcaaccccctgacaaaacacatagcctaccccttccttgtactatccctatgaggcataattataacaagctccatctgcctacgacaaacagacctaaaatcgctcattgcatactcttcaatcagccacatagccctcgtagtaacagccattctcatccaaaccccctgaagcttcaccggcgcagtcattctcataatcgcccacgggcttacatcctcattactattctgcctagcaaactcaaactacgaacgcactcacagtcgcatcataatcctctctcaaggacttcaaactctactcccactaatagctttttgatgacttctagcaagcctcgctaacctcgccttaccccccactattaacctactgggagaactctctgtgctagtaaccacgttctcctgatcaaatatcactctcctacttacaggactcaacatactagtcacagccctatactccctctacatatttaccacaacacaatggggctcactcacccaccacattaacaacataaaaccctcattcacacgagaaaacaccctcatgttcatacacctatcccccattctcctcctatccctcaaccccgacatcattaccgggttttcctcttgtaaatatagtttaaccaaaacatcagattgtgaatctgacaacagaggcttacgaccccttatttaccgagaaagctcacaagaactgctaactcatgcccccatgtctaacaacatggctttctcaacttttaaaggataacagctatccattggtcttaggccccaaaaattttggtgcaactccaaataaaagtaataaccatgcacactactataaccaccctaaccctgacttccctaattccccccatccttaccaccctcgttaaccctaacaaaaaaaactcatacccccattatgtaaaatccattgtcgcatccacctttattatcagtctcttccccacaacaatattcatgtgcctagaccaagaagttattatctcgaactgacactgagccacaacccaaacaacccagctctccctaagcttcaaactagactacttctccataatattcatccctgtagcattgttcgttacatggtccatcatagaattctcactgtgatatataaactcagacccaaacattaatcagttcttcaaatatctactcatcttcctaattaccatactaatcttagttaccgctaacaacctattccaactgttcatcggctgagagggcgtaggaattatatccttcttgctcatcagttgatgatacgcccgagcagatgccaacacagcagccattcaagcaatcctatacaaccgtatcggcgatatcggtttcatcctcgccttagcatgatttatcctacactccaactcatgagacccacaacaaatagcccttctaaacgctaatccaagcctcaccccactactaggcctcctcctagcagcagcaggcaaatcagcccaattaggtctccacccctgactcccctcagccatagaaggccccaccccagtctcagccctactccactcaagcactatagttgtagcaggaatcttcttactcatccgcttccaccccctagcagaaaatagcccactaatccaaactctaacactatgcttaggcgctatcaccactctgttcgcagcagtctgcgcccttacacaaaatgacatcaaaaaaatcgtagccttctccacttcaagtcaactaggactcataatagttacaatcggcatcaaccaaccacacctagcattcctgcacatctgtacccacgccttcttcaaagccatactatttatgtgctccgggtccatcatccacaaccttaacaatgaacaagatattcgaaaaataggaggactactcaaaaccatacctctcacttcaacctccctcaccattggcagcctagcattagcaggaatacctttcctcacaggtttctactccaaagaccacatcatcgaaaccgcaaacatatcatacacaaacgcctgagccctatctattactctcatcgctacctccctgacaagcgcctatagcactcgaataattcttctcaccctaacaggtcaacctcgcttccccacccttactaacattaacgaaaataaccccaccctactaaaccccattaaacgcctggcagccggaagcctattcgcaggatttctcattactaacaacatttcccccgcatcccccttccaaacaacaatccccctctacctaaaactcacagccctcgctgtcactttcctaggacttctaacagccctagacctcaactacctaaccaacaaacttaaaataaaatccccactatgcacattttatttctccaacatactcggattctaccctagcatcacacaccgcacaatcccctatctaggccttcttacgagccaaaacctgcccctactcctcctagacctaacctgactagaaaagctattacctaaaacaatttcacagcaccaaatctccacctccatcatcacctcaacccaaaaaggcataattaaactttacttcctctctttcttcttcccactcatcctaaccctactcctaatcacataacctattcccccgagcaatctcaattacaatatatacaccaacaaacaatgttcaaccagtaactactactaatcaacgcccataatcatacaaagcccccgcaccaataggatcctcccgaatcaaccctgacccctctccttcataaattattcagcttcctacactattaaagtttaccacaaccaccaccccatcatactctttcacccacagcaccaatcctacctccatcgctaaccccactaaaacactcaccaagacctcaacccctgacccccatgcctcaggatactcctcaatagccatcgctgtagtatatccaaagacaaccatcattccccctaaataaattaaaaaaactattaaacccatataacctcccccaaaattcagaataataacacacccgaccacaccgctaacaatcaatactaaacccccataaataggagaaggcttagaagaaaaccccacaaaccccattactaaacccacactcaacagaaacaaagcatacatcattattctcgcacggactacaaccacgaccaatgatatgaaaaaccatcgttgtatttcaactacaagaacaccaatgaccccaatacgcaaaactaaccccctaataaaattaattaaccactcattcatcgacctccccaccccatccaacatctccgcatgatgaaacttcggctcactccttggcgcctgcctgatcctccaaatcaccacaggactattcctagccatgcactactcaccagacgcctcaaccgccttttcatcaatcgcccacatcactcgagacgtaaattatggctgaatcatccgctaccttcacgccaatggcgcctcaatattctttatctgcctcttcctacacatcgggcgaggcctatattacggatcatttctctactcagaaacctgaaacatcggcattatcctcctgcttgcaactatagcaacagccttcataggctatgtcctcccgtgaggccaaatatcattctgaggggccacagtaattacaaacttactatccgccatcccatacattgggacagacctagttcaatgaatctgaggaggctactcagtagacagtcccaccctcacacgattctttacctttcacttcatcttgcccttcattattgcagccctagcaacactccacctcctattcttgcacgaaacgggatcaaacaaccccctaggaatcacctcccattccgataaaatcaccttccacccttactacacaatcaaagacgccctcggcttacttctcttccttctctccttaatgacattaacactattctcaccagacctcctaggcgacccagacaattataccctagccaaccccttaaacacccctccccacatcaagcccgaatgatatttcctattcgcctacacaattctccgatccgtccctaacaaactaggaggcgtccttgccctattactatccatcctcatcctagcaataatccccatcctccatatatccaaacaacaaagcataatatttcgcccactaagccaatcactttattgactcctagccgcagacctcctcattctaacctgaatcggaggacaaccagtaagctacccttttaccatcattggacaagtagcatccgtactatacttcacaacaatcctaatcctaataccaactatctccctaattgaaaacaaaatactcaaatgggcctgtccttgtagtataaactaatacaccagtcttgtaaaccggagatgaaaacctttttccaaggacaaatcagagaaaaagtctttaactccaccattagcacccaaagctaagattctaatttaaactattctctgttctttcatggggaagcagatttgggtaccacccaagtattgactcacccatcaacaaccgctatgtatttcgtacattactgccagccaccatgaatattgtacggtaccataaatacttgaccacctgtagtacataaaaacccaatccacatcaaaaccccctccccatgcttacaagcaagtacagcaatcaaccctcaactatcacacatcaactgcaactccaaagccacccctcacccactaggataccaacaaacctacccacccttaacagtacatagtacataaagccatttaccgtacatagcacattacagtcaaatcccttctcgtccccatggatgacccccctcagataggggtcccttgaccaccatcctccgtgaaatcaatatcccgcacaagagtgctactctcctcgctccgggcccataacacttgggggtagctaaagtgaactgtatccgacatctggttcctacttcagggtcataaagcctaaatagcccacacgttccccttaaataagacatcacgatg

SEQ ID NO: 3 - DNA, 522 bp, Homo sapiens
atgatgtatgctttgtttctgttgagtgtgggtttagtaatggggtttgtggggttttcttctaagccttctcctatttatgggggtttagtattgattgttagcggtgtggtcgggtgtgttattattctgaattttgggggaggttatatgggtttaatagtttttttaatttatttagggggaatgatggttgtctttggatatactacagcgatggctattgaggagtatcctgaggcatgagggtcaggggttgaggtcttggtgagtgttttagtggggttagcgatggaggtaggattggtgctgtgagtgaaagagtatgatggggtggtggttgtggtaaactttaatagtgtaggaagctgaataatttatgaaggagaggggtcagggttgattcgggaggatcctattggtgcgggggctttgtatgattatgggcgttgattagtagtagttactggttgaacattgtttgttggtgtatatattgtaattgagattgctcgggggaat

SEQ ID NO: 4 - DNA, 525 bp, Homo sapiens
atgatgtatgctttgtttctgttgagtgtgggtttagtaatggggtttgtggggttttcttctaagccttctcctatttatgggggtttagtattgattgttagcggtgtggtcgggtgtgttattattctgaattttgggggaggttatatgggtttaatagtttttttaatttatttagggggaatgatggttgtctttggatatactacagcgatggctattgaggagtatcctgaggcatgggggtcaggggttgaggtcttggtgagtgttttagtggggttagcgatggaggtaggattggtgctgtgggtgaaagagtatgatggggtggtggttgtggtaaactttaatagtgtaggaagctgaataatttatgaaggagaggggtcagggttgattcgggaggatcctattggtgcgggggctttgtatgattatgggcgttgattagtagtagttactggttgaacattgtttgttggtgtatatattgtaattgagattgctcgggggaatagg

SEQ ID NO: 5 - DNA, 516 bp, Mus musculus
atgaacaactatattttcgtcttaagttcattattcttggttggttgtcttgggttagcattaaagccttcacctatttatggaggtttaggtttaattgttagtgggtttgttggttgtttaatggttttagggtttggtggatcgttcttaggtttaatagttttcttaatttatttaggggggatgttggttgtgtttggatatacgactgctatagctactgaggaatatccagagacttgaggatctaactgattaattttgggtttcttagtattgggggtgattatagaggttttcttaatttgtgtgcttaattattatgatgaagttggagtaattaatcttgatggtttgggagattgattgatgtatgaggttgatgatgttggagttatgttggaaggagggattggggtagcggcaatatatagttgtgctacttgaatgatggtagtagctgggtgatctttgtttgcgggtattttcattattatcgagattactcgagat

SEQ ID NO: 6 - DNA, 519 bp, Mus musculus
atgaataattatatttttgttttaagttcattatttttggttggttgtcttgggttagcattaaagccttcacctatttatggaggtttaggtttaattgttagtgggtttgttggttgtttaatggttttagggtttggtggatcgtttttaggtttaatagtttttttaatttatttaggggggatgttggttgtgtttggatatacgactgctatagctactgaggaatatccagagacttggggatctaactgattaattttgggttttttagtattgggggtgattatagaggtttttttaatttgtgtgcttaattattatgatgaagttggagtaattaatcttgatggtttgggagattggttgatgtatgaggttgatgatgttggagttatgttggaaggagggattggggtagcggcaatatatagttgtgctacttgaatgatggtagtagctgggtgatctttgtttgcgggtatttttattattatcgagattactcgagattaa

SEQ ID NO: 7 - DNA, 11 bp, Artificial sequence
  Synthetic oligonucleotide
atcccccctaa

SEQ ID NO: 8 - DNA, 12 bp, Artificial sequence
  Synthetic oligonucleotide
atccccccctaa

SEQ ID NO: 9 - DNA, 10 bp, Artificial sequence
  Synthetic oligonucleotide
taaaaaaaat

SEQ ID NO: 10 - DNA, 11 bp, Artificial sequence
  Synthetic oligonucleotide
taaaaaaaaat

SEQ ID NO: 11 - DNA, 12 bp, Artificial sequence
  Synthetic oligonucleotide
taaaaaaaaaat

SEQ ID NO: 12 - DNA, 8 bp, Artificial sequence
  Synthetic oligonucleotide
tcccccct

SEQ ID NO: 13 - DNA, 9 bp, Artificial sequence
  Synthetic oligonucleotide
tccccccct

(19) United States
(12) Patent Application Publication
(10) Pub. No.: US 2021/0163898 A1
(43) Pub. Date: Jun. 3, 2021

(54) MITOCHONDRIAL RNA IMPORT FOR TREATING MITOCHONDRIAL DISEASE

(71) Applicant: THE CHILDREN'S HOSPITAL OF PHILADELPHIA

(72) Inventors: Atif TOWHEED, PHILADELPHIA, PA (US); Douglas C. WALLACE, PHILADELPHIA, PA (US); Michael J. PALLADINO, Philadelphia, PA (US)

(73) Assignees: THE CHILDREN'S HOSPITAL OF PHILADELPHIA, PHILADELPHIA, PA (US); UNIVERSITY OF PITTSBURGH - OF THE COMMONWEALTH SYSTEM OF HIGHER EDUCATION, Pittsburgh, PA (US)

(21) Appl. No.: 17/047,967

(22) PCT Filed: Apr. 16, 2019
(86) PCT No.: PCT/US2019/027676

§ 371 Date: Oct. 15, 2020

Related U.S. Application Data
(60) Provisional application No. 62/658,303, filed on Apr. 16, 2018.

(51) Int. Cl. C12N 9/02; C12N 15/86

(52) U.S. Cl. CPC C12N 9/0036; C12N 15/86; A61K 48/00; C12N 2840/102; C12Y 106/99003

(57) ABSTRACT

Disclosed is an RNA allotopic strategy to complement and genetically rescue mitochondrial gene defects. This approach can permit rescue of a mitochondrial DNA mutant by allotopic expression of a full-length recoded mitochondrial RNA that is transcribed in the nucleus, successfully imported into the mitochondria, and expressed there.

### PRIORITY CLAIM

This application claims benefit of priority to U.S. Provisional Application Ser. No. 62/658,303, filed Apr. 16, 2018, the entire contents of which are hereby incorporated by reference.

### BACKGROUND

### I. Field

The present disclosure relates to the fields of molecular biology, cell biology, genetics and medicine. More particularly, the disclosure relates to vectors and RNA that facilitate allotopic expression of proteins in mitochondria, and methods of use thereof. In particular, the disclosure relates to tRNA-containing vectors and related RNAs that are imported in mitochondria and efficiently translated.

### II. Related Art

Gene therapy targeted for mitochondrial DNA disorders face several challenges. Unlike nuclear genes which are present as diploids, cells contain numerous mitochondria ranging from 100-1000. Several strategies have been explored to target normal copy of mitochondrial genes into the mitochondrial matrix. However, mitochondrial double membrane is comparatively impermeable. Therefore, allotopic expression of mitochondrial-encoded proteins in the nucleus has a huge potential.

There are two strategies to allotopically express mitochondrial DNA (mtDNA) encoded genes. The first is to express mtDNA genes in the nucleus and have it translated in the cytosol. This translated protein has a mitochondrial targeting peptide sequence (MTS) which then delivers the normal copy of the gene to the mitochondrial membrane. This MTS based strategy has been commonly used and several groups have reported complementing mtDNA disorders in vitro (Manfredi et al., DOI: 10.1038/ng851, Bokori-Brown and Holt, 2006, Guy et al., 2002, Qi et al., 2007, Ellouse et al., 2008, Yu et al., 2012a,b) as well as in vivo (Qi et al., 2007, Ellouze et al., 2008, Yu et al., 2012a; 2012b; Yu et al., 2013). However, such approaches have had intrinsic challenges due to interspecies incompatibility or hydrophobic aggregations outside the mitochondrial membrane (McKenzie et al., 2003, Perales-Clemente at al., 2011). Whether such approach truly integrates the imported protein into a functional complex has been a major debate.

An alternate strategy is to target the gene transcript in the form of RNA (and not protein) into the mitochondrial matrix where it can be translated by the mitochondrial translation machinery instead of being translated in the cytosol. It has previously been shown that several species of RNA are naturally imported into the mitochondria. Previous studies have also utilized a strategy to tag these imported RNA/sequences to transport shorter or longer RNA sequences into the mitochondria (Kolesnikova et al., Science 2000, Wang et al., PNAS 2012 and Cell, Towheed et al., 2014). The first ever strategy to target a small tRNA sequence into the mitochondria was reported by Kolesnikova et al., Science 2000. More recently, using MERRF and MELAS cells Wang et al., demonstrated import of tRNA_{AAA}^{Lys} and tRNA_{UUR}^{Leu} conjugated with a mitochondrial targeted 20 nucleotide RP import sequence. mtRNA translation was found to be increased as a result of tRNA import into the mitochondria suggesting possible rescue of these mutant cells. Using the same RP sequence, Wang et al., also were able to demonstrate that a full-length mouse mitochondrial encoded gene COXII could be imported and translated within the mitochondria of Hela cells. However, efficient genetic rescue of a mitochondrial DNA mutant using allotopic RNA expression of a full-length mitochondrial mRNA has never been reported.

### SUMMARY

Thus, in accordance with the present disclosure, there is provided an RNA comprising, in a 5′ to 3′ order, a mitochondrial tRNA element, a first open reading frame, and a translation termination signal. The RNA may further comprise a mitochondrial signal sequence (mtRSS) located 5′ to said mitochondrial tRNA element. The RNA may further comprise a second open reading from downstream of said first open reading frame, and upstream of said translation termination signal. The RNA may further comprise a 3′ untranslated region (UTR).

The mitochondrial tRNA element may be a TRNE element, such as 5′-agggggttagttttgcgtattggggtcattggtgttcttgtagttgaaatacaacgatggtttttcatatcattggtcgtggttgtagtccgtgcgag aata-3′ (SEQ ID NO: 1). The mtRSS may be a 5S mtRSS. The first open reading frame may encode a mitochondrial protein. The second open reading frame may encode a detectable marker, such as a protein tag (e.g., FLAG) or a fluorescent protein. The translation termination signal may be TAA.

Also provided is a host cell comprising the RNA as described above.

In another embodiment, there is provided an expression cassette comprising, in a 5′ to 3′ order, an RNA polymerase II promoter, a mitochondrial tRNA RNE element, a first open reading frame, and a translation termination signal.

The expression construct may further comprise a mitochondrial signal sequence (mtRSS) located 5′ to said mitochondrial tRNA element. The expression construct may further comprise a second open reading from downstream of said first open reading frame, and upstream of said translation termination signal. The expression construct may further comprise a 3′ untranslated region (UTR).

The mitochondrial tRNA element may be a TRNE element, such as 5′-agggggttagttttgcgtattggggtcattggtgttcttgtagttgaaatacaacgatggtttttcatatcattggtcgtggttgtagtccgtgcgag aata-3′ (SEQ ID NO: 1). The mtRSS may be a 5S mtRSS. The first open reading frame may encode a mitochondrial protein. The second open reading frame may encode a detectable marker, such as a protein tag (e.g., FLAG) or a fluorescent protein. The translation termination signal may be TAA. The RNA polymerase II promoter (RNAPII) may be a eukaryotic RNAPII promoter, such as a chicken β actin promoter or a cytomegalovirus promoter. The expression cassette may be comprised in a selectable and/or replicable vector, such as a viral vector, such as a lentiviral vector, or an adeno-associated viral vector.

Also provided is a host cell comprising the expression cassette as described above.

Other embodiments include (a) a method of expressing an RNA in a cell comprising contacting a cell with an expression cassette as described above and culturing said cell under conditions supporting transcription and translation of an RNA encoded by said expression cassette, (b) a method of expressing an RNA in a cell comprising culturing said a host cell comprising an expression cassette as described above under conditions supporting transcription and translation of an RNA encoded by said expression cassette. The expression constructs may further comprise a second open reading from downstream of said first open reading frame, and upstream of said translation termination signal. The expression constructs may further comprise a 3′ untranslated region (UTR).

The mitochondrial tRNA element may be a TRNE element, such as 5′-agggggttagttttgcgtattggggtcattggtgttcttgtagttgaaatacaacgatggtttttcatatcattggtcgtggttgtagtccgtgcgag aata-3′ (SEQ ID NO: 1). The mtRSS may be a 5S mtRSS. The first open reading frame may encode a mitochondrial protein. The second open reading frame may encode a detectable marker, such as a protein tag (e.g., FLAG) or a fluorescent protein. The translation termination signal may be TAA. The RNA polymerase II promoter (RNAPII) may be a eukaryotic RNAPII promoter, such as a chicken β actin promoter or a cytomegalovirus promoter. The expression cassette may be comprised in a selectable and/or replicable vector, such as a viral vector, such as a lentiviral vector, or an adeno-associated viral vector.

In yet another embodiment, there is provided a method of complementing a defect in a mutated mitochondrial protein in a cell comprising contacting said cell with an expression cassette comprising, in a 5′ to 3′ order, an RNA Pol II promoter, a mitochondrial tRNA element, a first open reading frame encoding a non-mutant form of said mutated mitochondrial protein, and a translation termination signal. The expression construct may further comprise a second open reading from downstream of said first open reading frame, and upstream of said translation termination signal. The expression construct may further comprise a 3′ untranslated region (UTR).

The mitochondrial respiratory chain consists of four large protein complexes: I, II, III and IV (cytochrome c oxidase, or COX), ATP synthase, and two small molecules that ferry around electrons, coenzyme Q10 and cytochrome c. The respiratory chain is the final step in the energy-making process in the mitochondrion where most of the ATP is generated. Mitochondrial encephalomyopathies that can be caused by deficiencies in one or more of the specific respiratory chain complexes include MELAS, MERFF, Leigh's syndrome, KSS, Pearson, PEO, NARP, MILS and MNGIE. The mitochondrial respiratory chain is made up of proteins that come from both nuclear and mtDNA. Although only 13 of roughly 100 respiratory chain proteins come from the mtDNA, these 13 proteins contribute to every part of the respiratory chain except complex II, and 24 other mitochondrial genes are required just to manufacture those 13 proteins. Thus, a defect in either a nuclear gene or one of the 37 mitochondrial genes can cause the respiratory chain to break down. Depending on which cells are affected, symptoms may include loss of motor control, muscle weakness and pain, gastrointestinal disorders and swallowing difficulties, poor growth, cardiac disease, liver disease, diabetes, respiratory complications, seizures, visual/hearing problems, lactic acidosis, developmental delays and susceptibility to infection.

The mitochondrial tRNA element may be a TRNE element, such as 5′-agggggttagttttgcgtattggggtcattggtgttcttgtagttgaaatacaacgatggtttttcatatcattggtcgtggttgtagtccgtgcgag aata-3′ (SEQ ID NO: 1). The mtRSS may be a 5S mtRSS. The first open reading frame may encode a mitochondrial protein. The second open reading frame may encode a detectable marker, such as a protein tag (e.g., FLAG) or a fluorescent protein. The translation termination signal may be TAA. The RNA polymerase II promoter (RNAPII) may be a eukaryotic RNAPII promoter, such as a chicken β actin promoter or a cytomegalovirus promoter. The expression cassette may be comprised in a selectable and/or replicable vector, such as a viral vector, such as a lentiviral vector, or an adeno-associated viral vector.

In still a further embodiment, there is provided a method of complementing a defect in a mutated mitochondrial ND6 protein in a cell comprising contacting said cell with a expression cassette comprising, in a 5′ to 3′ order, an RNA Pol II promoter, a mitochondrial tRNA element, a first open reading frame encoding a non-mutant form of said mutated ND6 protein, and a translation termination signal. The expression construct may further comprise a second open reading from downstream of said first open reading frame, and upstream of said translation termination signal. The expression construct may further comprise a 3′ untranslated region (UTR). The mitochondrial tRNA element may be a TRNE element, such as 5′-agggggttagttttgcgtattggggtcattggtgttcttgtagttgaaatacaacgatggtttttcatatcattggtcgtggttgtagtccgtgcgag aata-3′ (SEQ ID NO: 1). The mtRSS may be a 5S mtRSS. The first open reading frame may encode a mitochondrial protein. The second open reading frame may encode a detectable marker, such as a protein tag (e.g., FLAG) or a fluorescent protein. The translation termination signal may be TAA. The RNA polymerase II promoter (RNAPII) may be a eukaryotic RNAPII promoter, such as a chicken β actin promoter or a cytomegalovirus promoter. The expression cassette may be comprised in a selectable and/or replicable vector, such as a viral vector, such as a lentiviral vector, or an adeno-associated viral vector.

Exemplary mitochondrial diseases include but are not limited to: Alpers Disease; Barth syndrome; p-oxidation defects; carnitine-acyl-carnitine deficiency; carnitine deficiency; co-enzyme Q10 deficiency; Complex I deficiency; Complex II deficiency; Complex III deficiency; Complex IV deficiency; Complex V deficiency; cytochrome c oxidase (COX) deficiency; Chronic Progressive External Ophthalmoplegia Syndrome (CPEO); CPT I Deficiency; CPT II deficiency; Glutaric Aciduria Type II; lactic acidosis; Long-Chain Acyl-CoA Dehydrongenase Deficiency (LCAD); LCHAD; mitochondrial cytopathy; mitochondrial DNA depletion; mitochondrial encephalopathy; mitochondrial myopathy; Mitochondrial Encephalomyopathy with Lactic Acidosis and Stroke like episodes (MELAS); Myoclonus Epilepsy with Ragged Red Fibers (MERRF); Maternally Inherited Leigh's Syndrome (MILS); Myogastrointestinal encephalomyopathy (MNGIE); Neuropathy, ataxia and retinitis pigmentosa (NARP); Leber's Hereditary Optic Neuropathy (LHON); Progressive external ophthalmoplegia (PEO); Pearson syndrome; Kearns-Sayre syndrome (KSS); Leigh's syndrome; intermittent dysautonomia; pyruvate carboxylase deficiency; pyruvate dehydrogenase deficiency; respiratory chain mutations and deletions; Short-Chain Acyl-GoA Dehydrogenase Deficiency (SCAD); SCHAD; and Very Long-Chain Acyl-CoA Dehydrongenase Deficiency (VLCAD); Pearson's Disease. Some mitochondrial diseases are a result of problems in the respiratory chain in the mitochondria.

The cell may be located in a subject, and said expression cassette is administered to said subject. The subject may have been diagnosed with a primary mitochondrial disease, such as Leber's Hereditary Optic Neuropathy (LHON). The method may further comprise administering a second LHON therapy to said subject. The method may further comprise administering said expression cassette at least a second time, such as chronically. The subject may be a human or non-human animal.

Thus, embodiments of the present disclosure are directed to treating a mitochondrial disease by introducing a mitochondrial gene-targeting vector. The present disclosure encompasses manipulating the mutant mitochondrial genome of the mammalian cell to treat diseases caused by mitochondrial genetic defects or abnormalities by supplementing a normal copy of the mitochondrial DNA encoded gene.

One embodiment of the present disclosure provides a method for restoring or increasing respiratory chain function in host cells including introducing a mitochondrial targeting vector comprising a first nucleic acid sequence comprising a TRNE element, directly or indirectly linked to a second nucleic acid sequence, which may be a wild-type mitochondrial DNA sequence or an altered mitochondrial DNA sequence.

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, for the method being employed to determine the value, or that exists among the study subjects. Such an inherent variation may be a variation of ±10% of the stated value.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

The patent or application file contains at least one drawing executed in color. Copies of this patent or patent application publication with color drawing(s) will be provided by the Office upon request and payment of the necessary fee.

FIGS. 1A-C. (FIG. 1A) Schematic of the vector expressing mitochondrial targeted recoded human and mouse mtND6 gene. Construct design for the novel vector to evaluate expression of ND6 in ND6 mutant cell line. Red stem loop structure: Mitochondrial RNA Import Signal Sequence (mtRSS), Blue: tRNAglu (TRNE) and green: recoded wild-type ND6 sequence, black: 3× Flag-tag. (FIG. 1B) ³²P radiolabeled mtRSS and mtRSS+TRNE+recoded mtND6 on a 3.5% 7M-urea gel. (FIG. 1C) Mitochondrial RNA import assay to evaluate importability of the various elements engineered in the rescue vector

FIGS. 2A-F. Screening the stable cell lines based on extra-cellular acidification rate. (FIG. 2A) Cell culture flasks with LM129 (wild-type control cells), LMJL2 (mtND6 frameshift mutant control cells) and LMJL2+HsND6 (rescued cells). (FIG. 2B) Extra-cellular acidification rate (ECAR) after plating equal number (135,000) of cells per sample using Seahorse (n=6, t-test). Evaluating production and utilization of various metabolites in LM129, LMJL2 and rescued cells (LMJL2+HsND6) in high glucose media (4.5 g/L). FIG. 2C) Lactate production rate (n=4). (FIG. 2D) Glucose utilization rate (n=4), (FIG. 2D) Glutamine. (FIG. 2F) Glutamate.

FIGS. 3A-D. Allotopically expressed mitochondrial imported recoded mtND6 alters mitochondrial content. (FIG. 3A) Transmission electron microscope images of LM129 (wild-type control cells), LMJL2 (mtND6 frameshift mutant control cells), LMJL2+MmND6 (rescued cells) and LMJL2+HsND6 (rescued cells), at magnification 15000×. Mutant cells display empty spaces within the cytosol as marked by yellow arrows. (FIG. 3B) Quantifying electron dense mitochondrial structures within the cytosolic boundaries in nucleated cells (n= or >10 for each sample, t-test). (FIG. 3C) Quantifying mitochondrial content by using mitochondrial localizing dye, Mitotracker using flow cytometer (n=3, t-test). (FIG. 3D) mtDNA copy number using a ratio of a mitochondrial (mtND6) gene and a housekeeping nuclear gene (actin) (n=3, t-test)

FIGS. 4A-D. (FIG. 4A) Restoration of Complex I activity and redox states in rescued cells. (FIG. 4B) Complex I-NQR assay. (FIG. 4C) Cellular redox state measured as NADH and flavo autofluorescence intensity within their inducible range (5 μM FCCP, 5 μM Antimycin A) using flow cytometry. Values towards 0 indicate maximally oxidized, towards 1 maximally reduced redox state. Error bars indicate standard error (n=4). One-way ANOVA using Bonferroni's multiple comparison test was used to evaluate significance. (FIG. 4D) NAD/NADH ratio measured in whole cells using HPLC.

FIGS. 5A-C. (FIG. 5A) Improvement of mitochondrial respiration post rescue of mtND6 mutant cell cybrids. (FIGS. 5B-C) High resolution respirometry showing routine, leak and ETS states of respiration, respiratory ratios are shown in a tabular form corresponding each cell line. (FIG. 5D) Oxygen consumption rate (OCR) measured using Seahorse using 750 uM FCCP for each line, n=6, 135,000 cells each well. Key order top to bottom is left to right in figure panels.

FIGS. 6A-I. ND6 mutation increases ₘCa²⁺ efflux rate to match decreased bioenergetics. The control (LM129), ND6 mutant (LMJL2), and ND6 rescue (LMJL2+HsND6) cybrids were permeabilized with digitonin (40 μM) and mitochondrial membrane potential and ₘCa²⁺ uptake was measured simultaneously. (FIG. 6A) Mean traces of permeabilized control (black), mutant (red) and rescue (green) cybrids loaded with mitochondrial membrane potential indicator, JC-1 (800 nM). (FIG. 6B) Mean traces of permeabilized cybrids loaded with the ratiometric Ca²⁺ indicator Fura2-FF (0.5 μM) and pulsed with 20 μM Ca²⁺ at 350 s to measure ₘCa²⁺ uptake, followed by addition of 1 μM Ru360 at 550 s to inhibit MCU-mediated Ca²⁺ uptake and 10 μM CGP37157 at 610 s to inhibit mitochondrial NCLX and 2 μM uncoupler CCCP at 750 s to dissipate mitochondrial membrane potential. (FIG. 6C) Quantification of basal mitochondrial membrane potential before addition of extra-mitochondrial Ca²⁺ bolus. Data represents Mean±SEM; **P<0.005; n=4-5. (FIG. 6D) Zoom of ₘCa²⁺ uptake from B as a measure of ₘCa²⁺ influx rate. (FIG. 6E) Zoom of ₘCa²⁺ efflux from B after addition of Ru360 to as a measure of ₘCa²⁺ efflux rate. (FIG. 6F) Zoom of mean [Ca²⁺]ₒᵤₜ traces from B after addition of CCCP as a measure of mCa²⁺ released. (FIGS. 6G-H) Quantification of ₘCa²⁺ influx (FIG. 6G) and efflux (FIG. 6H) rates as a measure of decrease in bath Ca²⁺ fluorescence. (FIG. 6I) Quantification of total mCa²⁺ released after CCCP addition. Data represents Mean±SEM; *P<0.05; **P<0.005; ns: non-significant n=4-5. Key order top to bottom is left to right in figure panels.

FIG. 7. Human mitochondrial DNA sequence ID N_012920.1 (SEQ ID NO: 2).

FIG. 8. Analysis of Control-Mutant ND6 and Allotopic Completed Cell Line. The first agarose gel is of RNA RCR products shows in the left panel that the expression of the allotopic human ND6 mRNA is only present in the transformant (JL2-hsND6) and not in the mouse control line (LM129) or in the mouse ND6 null cell line (JL2). The right panel shows that the RNA integrity is good, ANT1 is a mouse nuclear gene and ND6 is the mouse mtDNA ND6 gene.

FIG. 9. Mitochondrial Processing of Allotopic Transcript in Transformed Cells. The second agarose gel of RNA PCR products demonstrates the processing of the allotopic mRNA in the JL2-hsND6 transformat. It shows that the 5S import RNA sequence is cleaved off in the transformant implying that the imported mRNA is properly processed in the transformant.

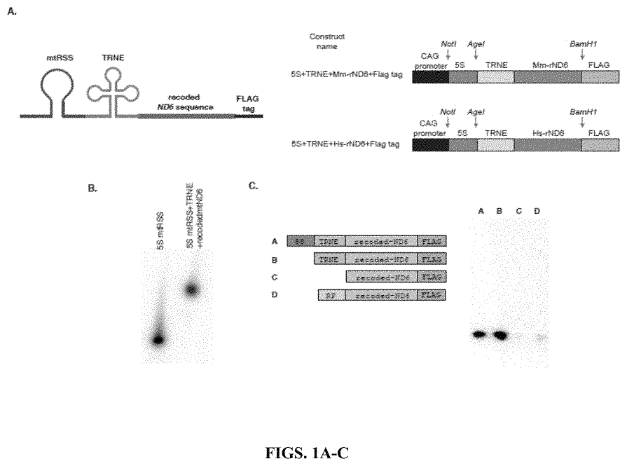

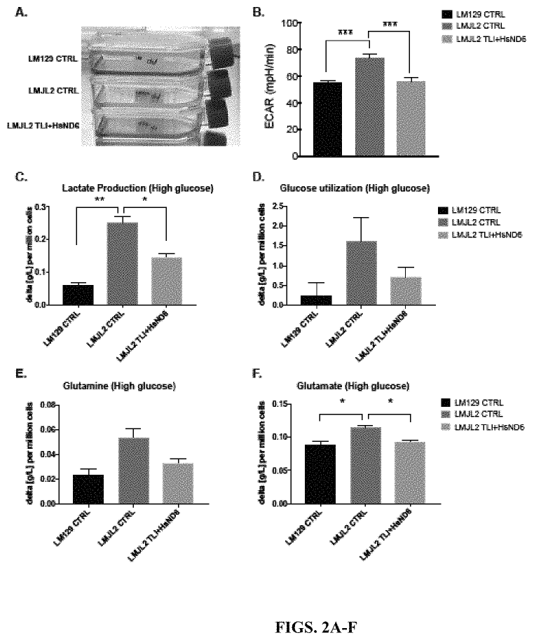

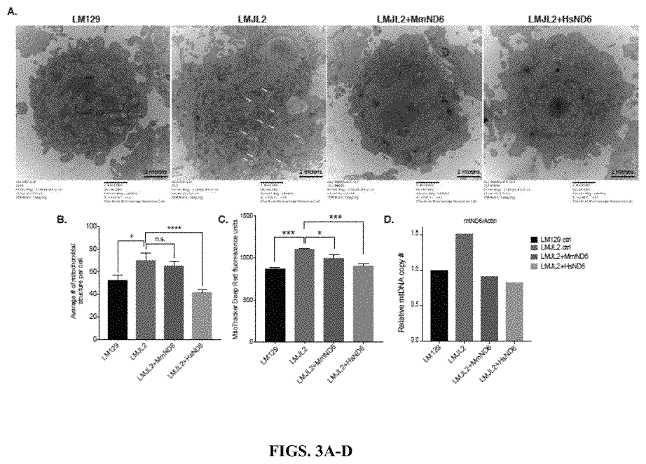

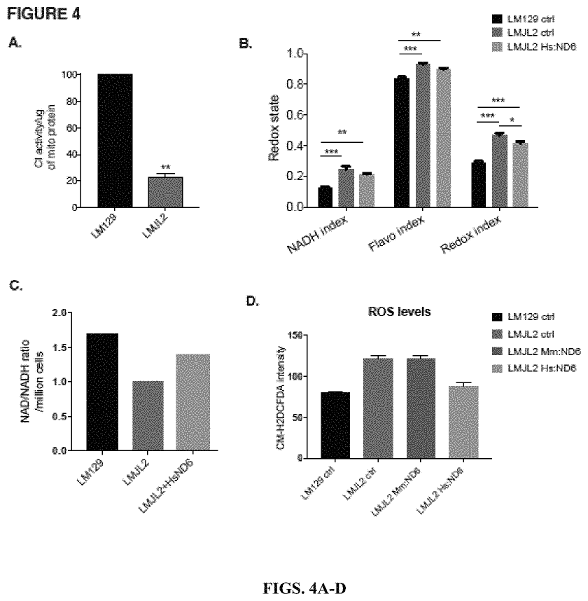

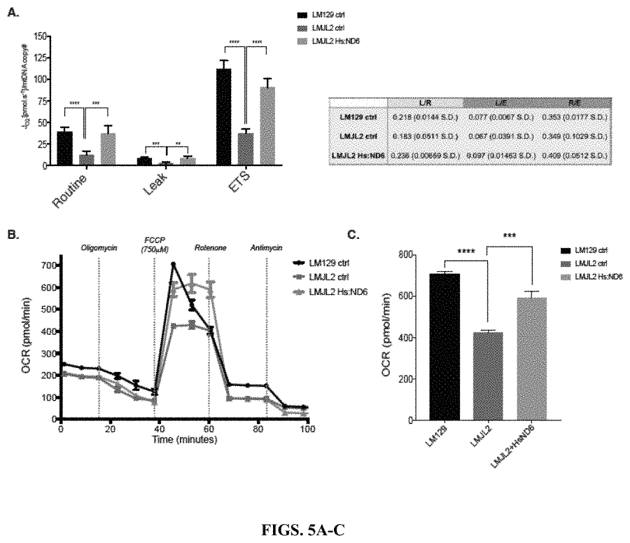

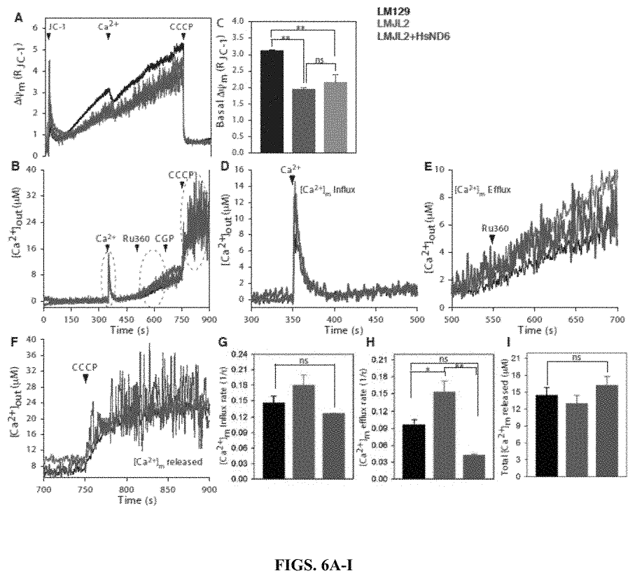

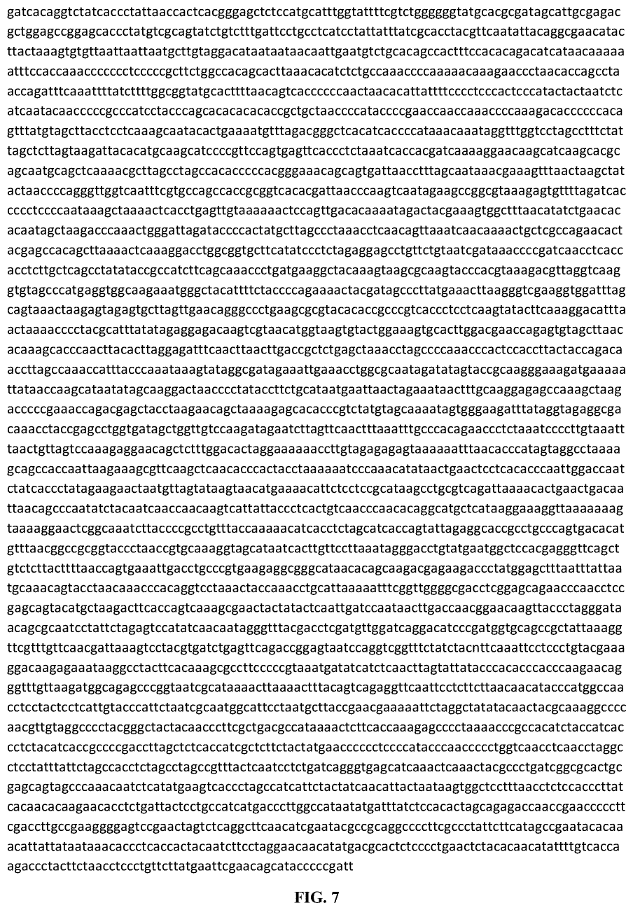

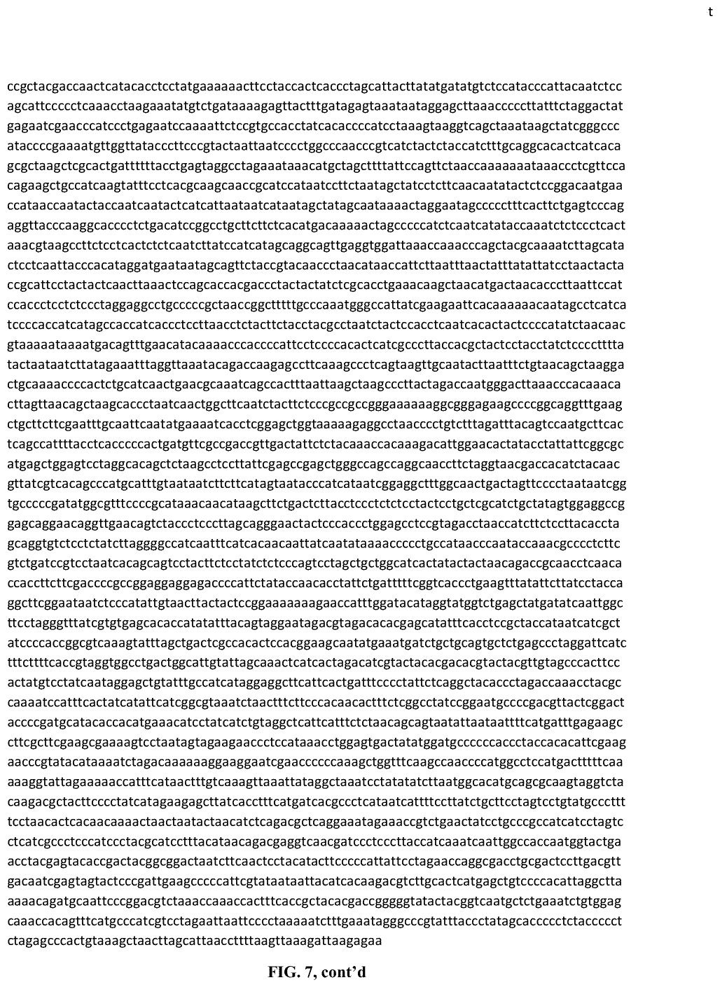

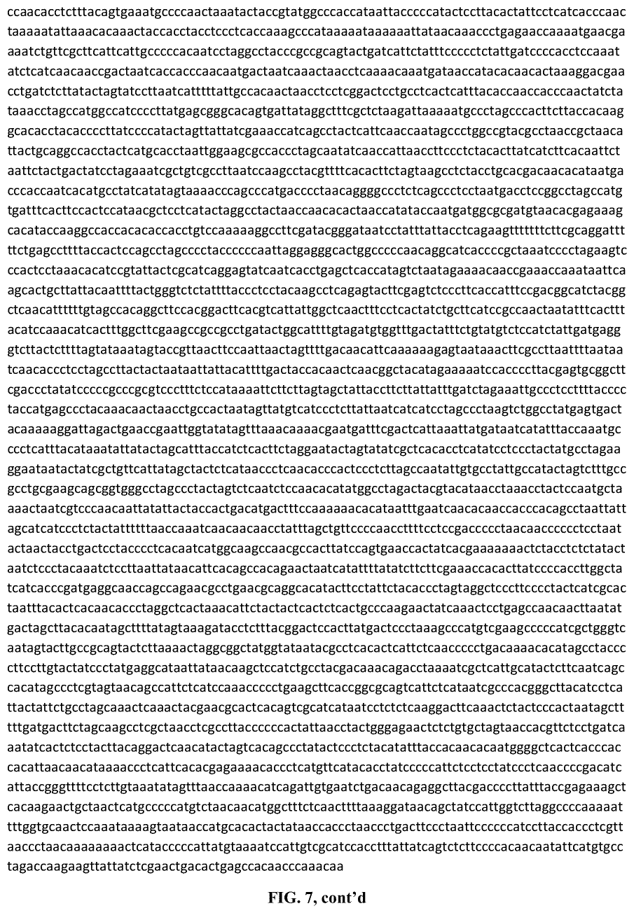

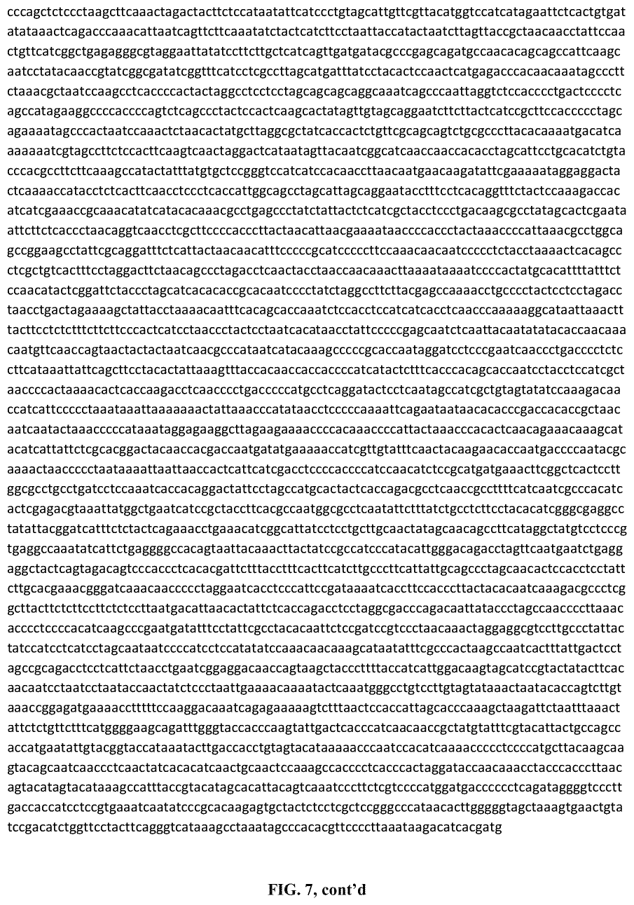

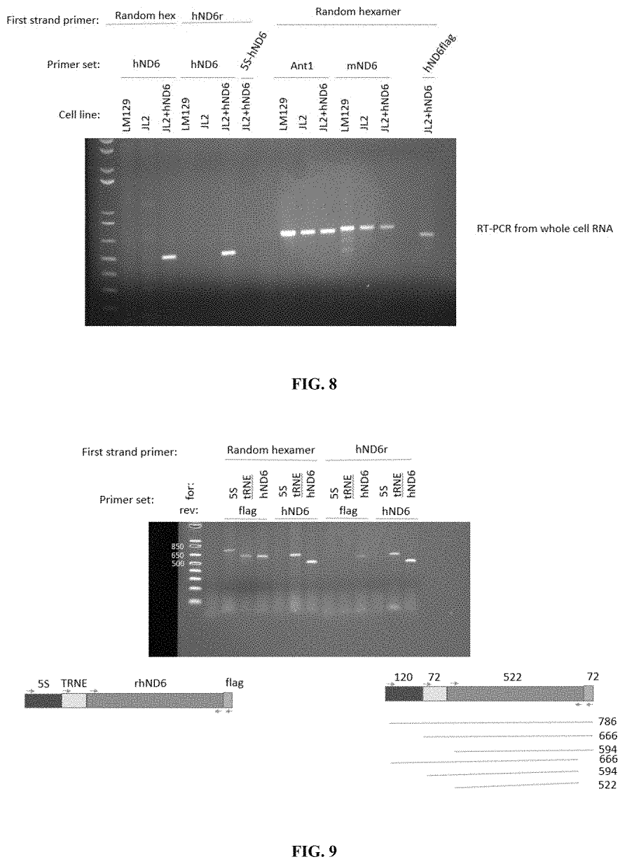

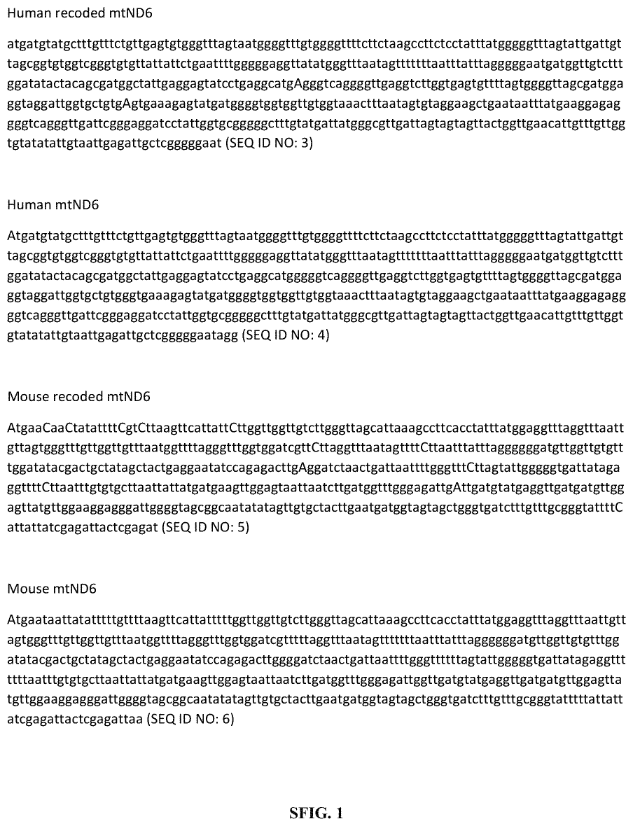

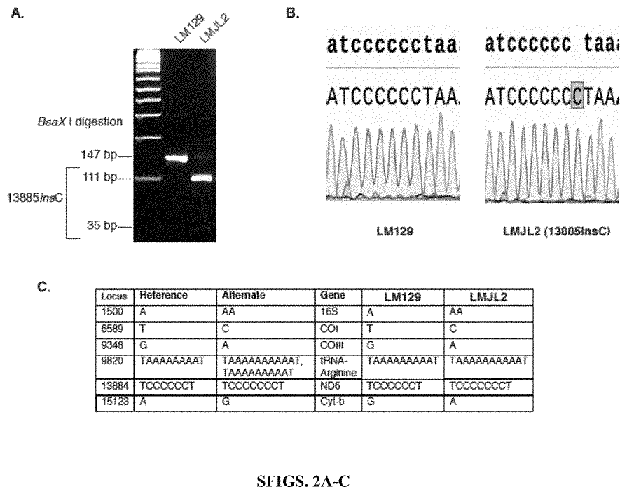

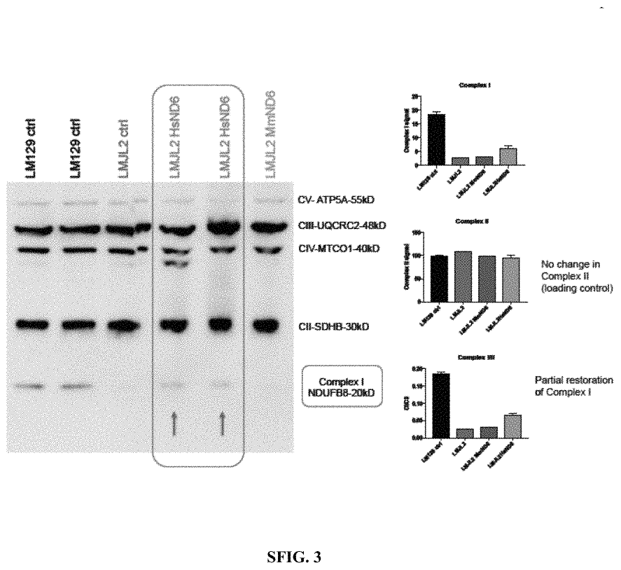

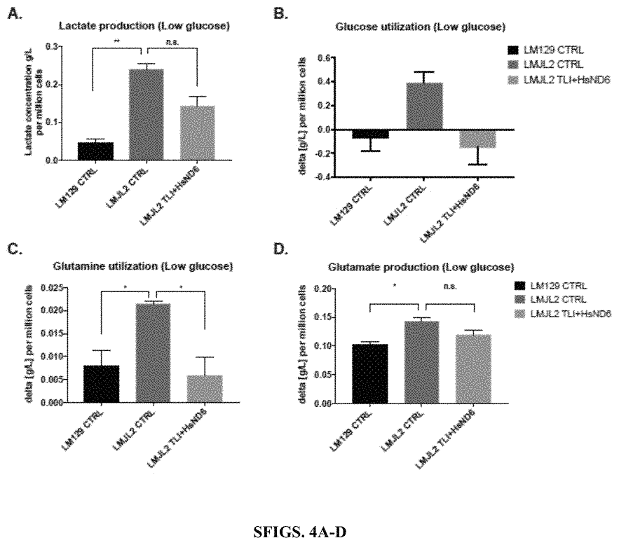

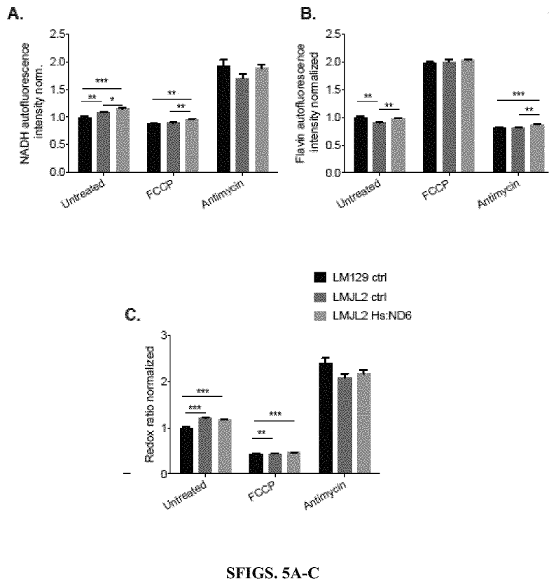

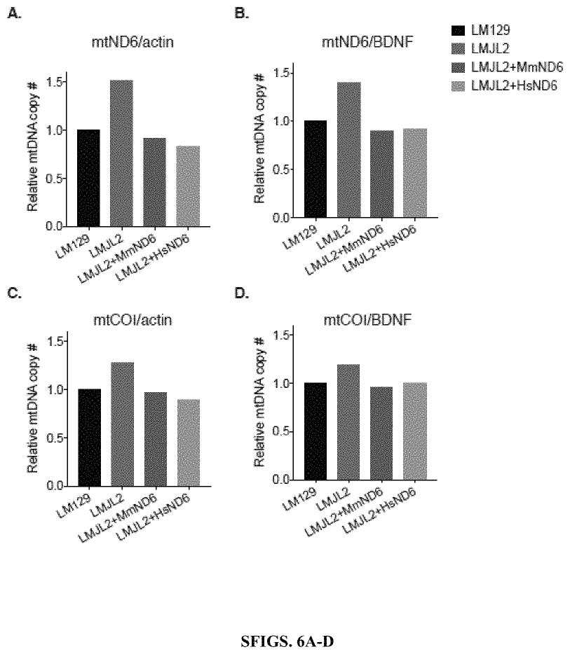

FIG. 1. Sequences of the engineered recoded mtND6 (mouse and human) aligned to naturally occurring mouse and human mtND6 sequences. Changes in the recoded mtND6 are shown in red and corresponding nucleotide is highlighted in green for each recoded and naturally occurring sequences respectively (Source: NCBI reference sequence NR_023363.1; Human).

SFIGS. 2A-C. (SFIG. 2A) Identification of mtDNA genotypes by Restriction enzyme digestion. 146-bp fragment around 13885 locus was PCR amplified using mismatched primers to engineer a restriction site for BsaX I enzyme that generates 110- and 36-bp fragments in the mutant mtDNA. (SFIG. 2B) Samples from each cell lines were confirmed using Sanger sequencing. (ATCCCCCCTAA=SEQ ID NO: 7; ATCCCCCCTAA=SEQ ID NO: 8) (SFIG. 2C) Homoplasmic loci using Next Generation Sequencing for (LM129) WT and (LMJL2) mutant cell cybrid lines. (TAAAAAAAAT=SEQ ID NO: 9; TAAAAAAAAAT=SEQ ID NO: 10; TAAAAAAAAAAT=SEQ ID NO: 11; TCCCCCCT=SEQ ID NO: 12; TCCCCCCCT=SEQ ID NO: 13)

SFIG. 3. Western Blot showing partial restoration of Complex I subunit NDUFB8 (20 kD) in the mito-cocktail antibody. Other mitochondrial complex subunits probed in the cocktail are CV (ATP5A-55 kD), CIII (UQCR2-48 kD), CIV (MTCO1-40 kD) and CII (SDHB-30 kD)

SFIG. 4A-D. Metabolite production and consumption rates in low glucose (1 g/L) media (n=4)

SFIGS. 5A-C. NADH (SFIG. 5A) and flavin (SFIG. 5B) autofluorescence intensities measured in untreated (Untreated), FCCP treated (5 μM) and Antimycin A treated (Antimycin, 5 μM) cells using flow cytometry. Results were normalized to the mean autofluorescence intensities of untreated, wild-type cells. The redox ratio of NADH/flavin autofluorescence intensity (SFIG. 5C) was calculated and normalized to untreated control. Error bars indicate standard deviation (n=4). One-way ANOVA using Bonferroni's multiple comparison test was used to evaluate significance.

SFIG. 6A-D. Mitochondrial DNA copy number using different combination of mitochondrial (mtND6 and mtCOI) and nuclear encoded (actin and BDNF) genes

### DETAILED DESCRIPTION

As discussed above, there is a need from improved technologies in the area of mitochondrial gene complementation. In this report, the inventors present an RNA allotopic strategy to complement and genetically rescue mtND6 gene in a previously characterized mtND6 frameshift (13885insC) mutation cell cybrid line. This is the first report to demonstrate the efficacy of rescue of a mitochondrial DNA mutant by using allotopic expression of a full-length “recoded” mitochondrial mRNA that is transcribed in the nucleus, successfully imported into the mitochondria, and there efficiently expressed. Such vectors are easily modified to contain a variety of open reading frames for allotopic expression of other genes either singularly or in combination, including each of the 13 mitochondrial genes, 22 tRNA genes and/or 2 rRNA genes. These and other aspects of the disclosure are set out in detail below.

### I. MITOCHONDRIA AND MITOCHONDRIAL GENE EXPRESSION

A. Mitochondrial Structure and Function

The mitochondrion is a double-membrane-bound organelle found in most eukaryotic organisms. Some cells in some multicellular organisms may however lack them (for example, mature mammalian red blood cells). A number of unicellular organisms, such as microsporidia, parabasalids, and diplomonads, have also reduced or transformed their mitochondria into other structures. To date, only one eukaryote, Monocercomonoides, is known to have completely lost its mitochondria. Mitochondria generate most of the cell's supply of adenosine triphosphate (ATP), used as a source of chemical energy.

Mitochondria are commonly between 0.75 and 3 μm in diameter but vary considerably in size and structure. Unless specifically stained, they are not visible. In addition to supplying cellular energy, mitochondria are involved in other tasks, such as signaling, cellular differentiation, and cell death, as well as maintaining control of the cell cycle and cell growth. Mitochondrial biogenesis is in turn temporally coordinated with these cellular processes. Mitochondria have been implicated in several human diseases, including mitochondrial disorders, cardiac dysfunction, heart failure and autism.

The number of mitochondria in a cell can vary widely by organism, tissue, and cell type. For instance, red blood cells have no mitochondria, whereas liver cells can have more than 2000. The organelle is composed of compartments that carry out specialized functions. These compartments or regions include the outer membrane, the intermembrane space, the inner membrane, and the cristae and matrix.

Mitochondrial DNA (mtDNA or mDNA) is the DNA located in mitochondria, cellular organelles within eukaryotic cells that convert chemical energy from food into a form that cells can use, adenosine triphosphate (ATP). Mitochondrial DNA is only a small portion of the DNA in a eukaryotic cell; most of the DNA can be found in the cell nucleus and, in plants and algae, also in plastids such as chloroplasts.

In humans, the 16,569 base pairs of mitochondrial DNA encode for only 37 genes. Human mitochondrial DNA was the first significant part of the human genome to be sequenced. In most species, including humans, mtDNA is inherited solely from the mother. Since animal mtDNA evolves faster than nuclear genetic markers, it represents a mainstay of phylogenetics and evolutionary biology. It also permits an examination of the relatedness of populations, and so has become important in anthropology and biogeography.

The transcription of mitochondrial DNA (mtDNA) is initiated from a small noncoding region, the D loop, and is regulated by nuclear-encoded proteins that are post-translationally imported into mitochondria. Mitochondrial RNAs are polycistronic precursor transcripts from both strands that are processed to release individual tRNAs, rRNAs and mRNAs. These RNAs undergo maturation (polyadenylation in some mitochondrial mRNA transcripts and CCA trinucleotide addition, to mRNAs and tRNAs, respectively). Using these essential set of 37 genes (13 protein coding genes, 2 rRNAs as well as 22 tRNAs) and macromolecules imported from the cytosol, this unique genetic system can translate the 13 protein mitochondrial DNA encoded genes and which get incorporated into the 5 complexes of the mitochondrial electron transport chain.

Nuclear and mitochondrial DNA are thought to be of separate evolutionary origin, with the mtDNA being derived from the circular genomes of the bacteria that were engulfed by the early ancestors of today's eukaryotic cells. This theory is called the endosymbiotic theory. Each mitochondrion is estimated to contain 2-10 mtDNA copies. In the cells of extant organisms, the vast majority of the proteins present in the mitochondria (numbering approximately 1100 different types in mammals) are coded for by nuclear DNA, but the genes for some, if not most, of them are thought to have originally been of bacterial origin, having since been transferred to the eukaryotic nucleus during evolution.

In most multicellular organisms, the mtDNA—or mitogenome—is organized as a circular, covalently closed, double-stranded DNA. For human mitochondrial DNA (and probably for that of metazoans in general), 100-10,000 separate copies of mtDNA are usually present per somatic cell (egg and sperm cells are exceptions). In mammals, each double-stranded circular mtDNA molecule consists of 15,000-17,000 base pairs. The two strands of mtDNA are differentiated by their nucleotide content, with a guanine-rich strand referred to as the heavy strand (or H-strand) and a cytosine-rich strand referred to as the light strand (or L-strand). The heavy strand encodes 28 genes, and the light strand encodes 9 genes for a total of 37 genes. Of the 37 genes, 13 are for proteins (polypeptides), 22 are for transfer RNA (tRNA) and two are for the small and large subunits of ribosomal RNA (rRNA). The human mitogenome contains overlapping genes (ATP8 and ATP6 as well as ND4L and ND4; see the human mitochondrial genome map), a feature that is rare in animal genomes. The 37-gene pattern is also seen among most metazoans, although in some cases one or more of these genes is absent and the mtDNA size range is greater.

B. Mitochondrial Genetic Disease

Mutations of mitochondrial DNA can lead to a number of illnesses including exercise intolerance and Kearns-Sayre syndrome (KSS), which causes a person to lose full function of heart, eye, and muscle movements. Some evidence suggests that they might be major contributors to the aging process and age-associated pathologies. Particularly in the context of disease, the proportion of mutant mtDNA molecules in a cell is termed heteroplasmy. The within-cell and between-cell distributions of heteroplasmy dictate the onset and severity of disease and are influenced by complicated stochastic processes within the cell and during development. Mutations in mitochondrial tRNAs can be responsible for severe diseases like the MELAS and MERRF syndromes (see below). In contrast, mutations in nuclear genes that encode proteins that mitochondria use can also contribute to mitochondrial diseases. These diseases do not follow mitochondrial inheritance patterns, but instead follow Mendelian inheritance patterns.

Leber's hereditary optic neuropathy (LHON). One particular mitochondrial disease is Leber's hereditary optic neuropathy (LHON), an eye disorder beginning in young adulthood (predominantly in males) that is characterized by progressive loss of central vision due to degeneration of the optic nerves and retina. Resulting from mitochondrial mutations in subunits of complex 1 of the oxidative phosphorylation chain, it manfiests by degeneration of retinal ganglion cells (RGCs) and their axons that leads to an acute or subacute loss of central vision.

In Northern European populations, about one in 9000 people carry one of the three primary LHON mutations. There is a prevalence of between 1:30,000 and 1:50,000 in Europe; for example, it affects 1 in 50,000 people in Finland. The LHON ND4 G11778A mutation dominates as the primary mutation in most of the world with 70% of Northern European cases and 90% of Asian cases. Due to a “founder” effect, the LHON ND6 T14484C mutation accounts for 86% of LHON cases in Quebec, Canada.

More than 50 percent of males with a mutation and more than 85 percent of females with a mutation never experience vision loss or related medical problems. The particular mutation type may predict the likelihood of penetrance, severity of illness and probability of vision recovery in the affected. As a rule of thumb, a woman who harbors a homoplasmic primary LHON mutation has a ˜40% risk of having an affected son and a ˜10% risk of having an affected daughter.

Additional factors may determine whether a person develops the signs and symptoms of this disorder. Environmental factors such as smoking and alcohol use may be involved, although studies of these factors have produced conflicting results. Researchers are also investigating whether changes in additional genes, particularly genes on the X chromosome, contribute to the development of signs and symptoms. The degree of heteroplasmy, the percentage of mitochondria which have mutant alleles, may play a role. Patterns of mitochondrial alleles called haplogroup may also affect expression of mutations.

LHON is only transmitted through the mother, as it is primarily due to mutations in the mitochondrial (not nuclear) genome, and only the egg contributes mitochondria to the embryo. LHON is usually due to one of three pathogenic mitochondrial DNA (mtDNA) point mutations. These mutations are at nucleotide positions 11778 G to A, 3460 G to A and 14484 T to C, respectively in the ND4, ND1 and ND6 subunit genes of complex I of the oxidative phosphorylation chain in mitochondria. Men cannot pass on the disease to their offspring.

Clinically, there is an acute onset of visual loss, first in one eye, and then a few weeks to months later in the other. Onset is usually young adulthood, but age range at onset from 7-75 is reported. The age of onset is slightly higher in females (range 19-55 years: mean 31.3 years) than males (range 15-53 years: mean 24.3). The male to female ratio varies between mutations: 3:1 for 3460 G>A, 6:1 for 11778 G>A and 8:1 for 14484 T>C.

The disease typically evolves to very severe optic atrophy and a permanent decrease of visual acuity. Both eyes become affected either simultaneously (25% of cases) or sequentially (75% of cases) with a median inter-eye delay of 8 weeks. Rarely only one eye may be affected. In the acute stage, lasting a few weeks, the affected eye demonstrates an edematous appearance of the nerve fiber layer especially in the arcuate bundles and enlarged or telangiectatic and tortuous peripapillary vessels (microangiopathy). The main features are seen on fundus examination, just before or subsequent to the onset of visual loss. A pupillary defect may be visible in the acute stage as well. Examination reveals decreased visual acuity, loss of color vision and a cecocentral scotoma on visual field examination.

“LHON Plus” is a name given to a rare variant of the disorder with eye disease together with other conditions. The symptoms of this higher form of the disease include loss of the brain's ability to control the movement of muscles, tremors, and cardiac arrhythmia. Many cases of LHON plus have been comparable to multiple sclerosis because of the lack of muscular control.

LHON is a condition related to changes in mitochondrial DNA. Although most DNA is packaged in chromosomes within the nucleus, mitochondria have a distinct mitochondrial genome composed of mtDNA. Mutations in the MT-ND1, MT-ND4, MT-ND4L, and MT-ND6 genes cause LHON. These genes code for the NADH dehydrogenase protein involved in the normal mitochondrial function of oxidative phosphorylation. Oxidative phosphorylation uses a series of four large multienzyme complexes, which are all embedded in the inner mitochondrial membrane to convert oxygen and simple sugars to energy. Mutations in any of the genes disrupt this process to cause a variety of syndromes depending on the type of mutation and other factors. It remains unclear how these genetic changes cause the death of cells in the optic nerve and lead to the specific features of LHON.

The eye pathology is limited to the retinal ganglion cell layer especially the maculopapillary bundle. Degeneration is evident from the retinal ganglion cell bodies to the axonal pathways leading to the lateral geniculate nuclei. Experimental evidence reveals impaired glutamate transport and increased reactive oxygen species (ROS) causing apoptosis of retinal ganglion cells. Also, experiments suggest that normal non LHON affected retinal ganglion cells produce less of the potent superoxide radical than other normal central nervous system neurons. Viral vector experiments which augment superoxide dismutase 2 in LHON cybrids or LHON animal models or use of exogenous glutathione in LHON cybrids have been shown to rescue LHON affected retinal ganglion cells from apoptotic death. These experiments may in part explain the death of LHON affected retinal ganglion cells in preference to other central nervous system neurons which also carry LHON affected mitochondria.

Without a known family history of LHON, the diagnosis usually requires a neuro-ophthalmological evaluation and blood testing for mitochondrial DNA assessment. It is important to exclude other possible causes of vision loss and important associated syndromes such as heart electrical conduction system abnormalities. The prognosis for those affected left untreated is almost always that of continued significant visual loss in both eyes. Regular corrected visual acuity and perimetry checks are advised for follow up of affected individuals. There is beneficial treatment available for some cases of this disease especially for early onset disease. Also, experimental treatment protocols are in progress. Genetic counselling should be offered. Health and lifestyle choices should be reassessed particularly in light of toxic and nutritional theories of gene expression. Vision aid and work rehabilitation should be used to assist in maintaining employment.

For those who are carriers of a LHON mutation, preclinical markers may be used to monitor progress. For example, fundus photography can monitor nerve fiber layer swelling. Optical coherence tomography can be used for more detailed study of retinal nerve fiber layer thickness. Red green color vision testing may detect losses. Contrast sensitivity may be diminished. There could be an abnormal electroretinogram or visual evoked potentials. Neuron-specific enolase and axonal heavy chain neurofilament blood markers may predict conversion to affected status. Cyanocobalamin (a form of B12) may also be used.

Avoiding optic nerve toxins is generally advised, especially tobacco and alcohol. Certain prescription drugs are known to be a potential risk, so all drugs should be treated with suspicion and checked before use by those at risk. Ethambutol, in particular, has been implicated as triggering visual loss in carriers of LHON. In fact, toxic and nutritional optic neuropathies may have overlaps with LHON in symptoms, mitochondrial mechanisms of disease and management. Of note, when a patient carrying or suffering from LHON or toxic/nutritional optic neuropathy suffers a hypertensive crisis as a possible complication of the disease process, nitroprusside (trade name: Nipride) should not be used due to increased risk of optic nerve ischemia in response to this anti-hypertensive in particular.

Idebenone, a short-chain benzoquinone that interacts with the mitochondrial electron transport chain to enhance cellular respiration, was first shown in a small placebo-controlled trial to have modest benefit in about half of patients, and subsequent larger trials confirmed the benefits of idebenone. Idebenone, combined with avoidance of smoke and limitation of alcohol intake, is therefore the preferred standard treatment protocol for patients affected by LHON. It is believed to allow electrons to bypass the dysfunctional complex I. People most likely to respond best were those treated early in onset. α-Tocotrienol-quinone, a vitamin E metabolite, has had some success in small open label trials in reversing early onset vision loss.

There are various treatment approaches which have had early trials or are proposed, none yet with convincing evidence of usefulness or safety for treatment or prevention including brimonidine, minocycline, curcumin, glutathione, near infrared light treatment, and viral vector techniques.

“Three-person in vitro fertilization” is a proof of concept research technique for preventing mitochondrial disease in developing human fetuses. So far, viable macaque monkeys have been produced. But ethical and knowledge hurdles remain before use of the technique in humans is established.

Other mitochondrial disease. Other examples of mitochondrial diseases include:
Mitochondrial myopathy
Diabetes mellitus and deafness (DAD) (this combination at an early age can be due to mitochondrial disease; diabetes mellitus and deafness can be found together for other reasons
Leigh syndrome, subacute sclerosing encephalopathy (after normal development the disease usually begins late in the first year of life, although onset may occur in adulthood; a rapid decline in function occurs and is marked by seizures, altered states of consciousness, dementia, ventilatory failure)
Neuropathy, ataxia, retinitis pigmentosa, and ptosis (NARP) (progressive symptoms as described in the acronym; dementia)
Myoneurogenic gastrointestinal encephalopathy (MNGIE) (gastrointestinal pseudo-obstruction; neuropathy)
Myoclonic Epilepsy with Ragged Red Fibers (MERRF) (progressive myoclonic epilepsy
“Ragged Red Fibers” are clumps of diseased mitochondria that accumulate in the subsarcolemmal region of the muscle fiber and appear when muscle is stained with modified Gömöri trichrome stain; short stature; hearing loss; lactic acidosis; exercise intolerance)
Mitochondrial myopathy, encephalomyopathy, lactic acidosis, stroke-like symptoms (MELAS)
mtDNA depletion (mitochondrial neurogastrointestinal encephalomyopathy (MNGIE))

Other examples of mitochondrial diseases relevant to the present disclosure are set forth in the Summary presented above.

### II. MITOCHONDRIAL TARGETING EXPRESSION VECTORS

The present inventors have developed and successfully evaluated vectors capable of facilitating allotopic expression of mitochondrial targeted RNA. A key feature of these constructs is a mitochondrial tRNA element. In some embodiments, the vector comprises in the 5′ to 3′ direction, a promoter sequence, the mitochondrial tRNA element, and an open reading frame (ORF) encoding a protein, a termination sequence, and optionally marker gene and/or a 3′UTR. The vector may further comprise a mitochondrial targeting signal sequence (mtRSS) located 5′ to the tRNA element.

In a specific embodiment, the mitochondrial tRNA element is TRNE. In more specific embodiments, TRNE comprise a 33 bp pre-tRNA^{Glu} and 69 bp mitochondrial DNA tRNA^{Glu} (TRNE) sequences (mouse mitochondrial DNA sequence ID NC-005089.1, loci i14071 . . . 14172; human mitochondrial DNA sequence (ID N_012920.1, loci 14674 . . . 14775). Other mitochondrial tRNA sequences include can be used as substitutes for TRNE.

The promoter sequence can be any nucleic acid sequence that is recognized by RNA polymerase (RNAPII) to initiate transcription. In some embodiments, the RNAPII promoter sequence is a CAG promoter sequence or CMV promoter.

The termination sequence can be any nucleic acid sequence that RNAPII recognizes as a transcription termination sequence.

In some embodiments of the disclosed vectors, at least one codon of the ORF is modified such that the protein can be translated in mitochondria but not in the cytosol. For example, the codon can be modified to introduce a premature stop codon to prevent expression of the protein (or expression of the full-length protein) in the cytosol, where the same codon is not read as a stop codon in the mitochondria, for example TGA. This effect takes advantage of codon differences between the cytosol and mitochondria, for example, where the codon of the ORF is modified to contain a premature stop codon if translated in the cytosol, and a tryptophan codon is translated in mitochondria.

The mitochondrial targeting signal sequence can be any nucleic acid sequence (RNA sequence in this case) that localizes in mitochondria. One example is the 5S signal sequences as 5S is the most abundantly localized RNA in the mitochondria. Other mitochondrial targeting sequences could be a tRNA sequence (either coded by the mitochondrial DNA or nuclear DNA), MRP, RNP either in full or truncated version that determines importability into the mitochondria.

The disclosed vectors can include an ORF encoding any protein of interest. In some embodiments, the ORF encodes a protein encoded by a mitochondrial gene. In some examples, the protein is encoded by the ND1, ND2, ND3, ND4, ND4L, ND5, ND6, CYTB, COX1, COX2, COX3, ATP6 or ATP8 gene. In other embodiments, the ORF encodes a reporter protein, such as a fluorescent protein such as GFP, a FLAG tag, biotin tag, etc.

Also provided are recombinant RNA molecules produced by expression of a vector as disclosed herein. Such RNA molecules would include, in a 5′ to 3′ order, an mtRSS, a tRNA element, an ORF, and a stop codon, optionally further including a marker coding segment or a 3′UTR.

### III. METHODS OF TRANSFORMING MITOCHONDRIA

There are a number of ways in which expression vectors may be introduced into cells. In certain embodiments of the disclosure, the expression construct comprises a virus or engineered construct derived from a viral genome. The ability of certain viruses to enter cells via receptor-mediated endocytosis, to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). The first viruses used as gene vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986). These have a relatively low capacity for foreign DNA sequences and have a restricted host spectrum. Furthermore, their oncogenic potential and cytopathic effects in permissive cells raise safety concerns. They can accommodate only up to 8 kB of foreign genetic material but can be readily introduced in a variety of cell lines and laboratory animals (Nicolas and Rubenstein, 1988; Temin, 1986).

One of method for in vivo delivery involves the use of an adenovirus expression vector. “Adenovirus expression vector” is meant to include those constructs containing adenovirus sequences sufficient to (a) support packaging of the construct and (b) to express an antisense polynucleotide that has been cloned therein. In this context, expression does not require that the gene product be synthesized.

The expression vector comprises a genetically engineered form of adenovirus. Knowledge of the genetic organization of adenovirus, a 36 kB, linear, double-stranded DNA virus, allows substitution of large pieces of adenoviral DNA with foreign sequences up to 7 kB (Grunhaus and Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage. So far, adenoviral infection appears to be linked only to mild disease such as acute respiratory disease in humans.

Adenovirus is particularly suitable for use as a gene transfer vector because of its mid-sized genome, ease of manipulation, high titer, wide target cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP, (located at 16.8 m.u.) is particularly efficient during the late phase of infection, and all the mRNA's issued from this promoter possess a 5′-tripartite leader (TPL) sequence which makes them preferred mRNA's for translation.

Generation and propagation of the current adenovirus vectors, which are replication deficient, depend on a unique helper cell line, designated 293, which was transformed from human embryonic kidney cells by Ad5 DNA fragments and constitutively expresses E1 proteins (Graham et al., 1977). Since the E3 region is dispensable from the adenovirus genome (Jones and Shenk, 1978), the current adenovirus vectors, with the help of 293 cells, carry foreign DNA in either the E1, the D3 or both regions (Graham and Prevec, 1991). In nature, adenovirus can package approximately 105% of the wild-type genome (Ghosh-Choudhury et al., 1987), providing capacity for about 2 extra kb of DNA. Combined with the approximately 5.5 kb of DNA that is replaceable in the E1 and E3 regions, the maximum capacity of the current adenovirus vector is under 7.5 kb, or about 15% of the total length of the vector. More than 80% of the adenovirus viral genome remains in the vector backbone and is the source of vector-borne cytotoxicity. Also, the replication deficiency of the E1-deleted virus is incomplete.

Adenovirus vectors have been used in eukaryotic gene expression (Levrero et al., 1991; Gomez-Foix et al., 1992) and vaccine development (Grunhaus and Horwitz, 1992; Graham and Prevec, 1991). Recently, animal studies suggested that recombinant adenovirus could be used for gene therapy (Stratford-Perricaudet and Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotactic inoculation into the brain (Le Gal La Salle et al., 1993).

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

A novel approach designed to allow specific targeting of retrovirus vectors was recently developed based on the chemical modification of a retrovirus by the chemical addition of lactose residues to the viral envelope. This modification could permit the specific infection of hepatocytes via sialoglycoprotein receptors.

A different approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

There are certain limitations to the use of retrovirus vectors in all aspects of the present disclosure. For example, retrovirus vectors usually integrate into random sites in the cell genome. This can lead to insertional mutagenesis through the interruption of host genes or through the insertion of viral regulatory sequences that can interfere with the function of flanking genes (Varmus et al., 1981). Another concern with the use of defective retrovirus vectors is the potential appearance of wild-type replication-competent virus in the packaging cells. This can result from recombination events in which the intact-sequence from the recombinant virus inserts upstream from the gag, pol, env sequence integrated in the host cell genome. However, new packaging cell lines are now available that should greatly decrease the likelihood of recombination (Markowitz et al., 1988; Hersdorffer et al., 1990).

Other viral vectors may be employed as expression constructs in the present disclosure. Vectors derived from viruses such as vaccinia virus (Ridgeway, 1988; Baichwal and Sugden, 1986; Coupar et al., 1988) adeno-associated virus (AAV) (Ridgeway, 1988; Baichwal and Sugden, 1986; Hermonat and Muzycska, 1984) and herpesviruses may be employed. They offer several attractive features for various mammalian cells (Friedmann, 1989; Ridgeway, 1988; Baichwal and Sugden, 1986; Coupar et al., 1988; Horwich et al., 1990).

In order to effect expression of sense or antisense gene constructs, the expression construct must be delivered into a cell. This delivery may be accomplished in vitro, as in laboratory procedures for transforming cells lines, or in vivo or ex vivo, as in the treatment of certain disease states. One mechanism for delivery is via viral infection where the expression construct is encapsidated in an infectious viral particle.

Several non-viral methods for the transfer of expression constructs into cultured mammalian cells also are contemplated by the present disclosure. These include calcium phosphate precipitation (Graham and Van Der Eb, 1973; Chen and Okayama, 1987; Rippe et al., 1990) DEAE-dextran (Gopal, 1985), electroporation (Tur-Kaspa et al., 1986; Potter et al., 1984), direct microinjection (Harland and Weintraub, 1985), DNA-loaded liposomes (Nicolau and Sene, 1982; Fraley et al., 1979) and lipofectamine-DNA complexes, cell sonication (Fechheimer et al., 1987), gene bombardment using high velocity microprojectiles (Yang et al., 1990), and receptor-mediated transfection (Wu and Wu, 1987; Wu and Wu, 1988). Some of these techniques may be successfully adapted for in vivo or ex vivo use.

In yet another embodiment of the disclosure, the expression construct may simply consist of naked recombinant DNA, naked recombinant RNA or plasmids. Transfer of the construct may be performed by any of the methods mentioned above which physically or chemically permeabilize the cell membrane. This is particularly applicable for transfer in vitro but it may be applied to in vivo use as well. Dubensky et al. (1984) successfully injected polyomavirus DNA in the form of calcium phosphate precipitates into liver and spleen of adult and newborn mice demonstrating active viral replication and acute infection. Benvenisty and Neshif (1986) also demonstrated that direct intraperitoneal injection of calcium phosphate-precipitated plasmids results in expression of the transfected genes. It is envisioned that DNA encoding a gene of interest may also be transferred in a similar manner in vivo and express the gene product.

In still another embodiment of the disclosure for transferring a naked DNA expression construct into cells may involve particle bombardment. This method depends on the ability to accelerate DNA-coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

Selected organs including the liver, skin, and muscle tissue of rats and mice have been bombarded in vivo (Yang et al., 1990; Zelenin et al., 1991). This may require surgical exposure of the tissue or cells, to eliminate any intervening tissue between the gun and the target organ, i.e., ex vivo treatment. Again, DNA encoding a particular gene may be delivered via this method and still be incorporated by the present disclosure.

In a further embodiment of the disclosure, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated are lipofectamine-DNA complexes.

Liposome-mediated nucleic acid delivery and expression of foreign DNA in vitro has been very successful. Wong et al., (1980) demonstrated the feasibility of liposome-mediated delivery and expression of foreign DNA in cultured chick embryo, HeLa and hepatoma cells. Nicolau et al., (1987) accomplished successful liposome-mediated gene transfer in rats after intravenous injection. A reagent known as Lipofectamine 2000™ is widely used and commercially available.

In certain embodiments of the disclosure, the liposome may be complexed with a hemagglutinating virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear non-histone chromosomal proteins (HMG-1) (Kato et al., 1991). In yet further embodiments, the liposome may be complexed or employed in conjunction with both HVJ and HMG-1. In that such expression constructs have been successfully employed in transfer and expression of nucleic acid in vitro and in vivo, then they are applicable for the present disclosure. Where a bacterial promoter is employed in the DNA construct, it also will be desirable to include within the liposome an appropriate bacterial polymerase.

Other expression constructs which can be employed to deliver a nucleic acid encoding a particular gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu, 1993).

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) (Wu and Wu, 1987) and transferrin (Wagner et al., 1990). Recently, a synthetic neoglycoprotein, which recognizes the same receptor as ASOR, has been used as a gene delivery vehicle (Ferkol et al., 1993; Perales et al., 1994) and epidermal growth factor (EGF) has also been used to deliver genes to squamous carcinoma cells (Myers, EPO 0273085).

Further provided herein are isolated host cells comprising vectors as disclosed herein.

Further provided herein is a method of targeting a recombinant RNA molecule to mitochondria of a cell, comprising contacting the cell with a transfer vector as disclosed herein, wherein expression of the vector in the cell produces the recombinant RNA molecule which is targeted to mitochondria. In some embodiments, the method is an in vitro method. In other embodiments, the method is an in vivo method.

Also provided herein is a method of treating a disease caused by a mutation in a mitochondrial gene. In some embodiments, the method includes selecting a subject with a disease caused by the mutation in the mitochondrial gene and administering to the subject a therapeutically effective amount of a vector disclosed herein.

### IV. PHARMACEUTICAL FORMULATIONS

Where clinical applications are contemplated, pharmaceutical compositions will be prepared in a form appropriate for the intended application. Generally, this will entail preparing compositions (e.g., expression vector) that are essentially free of pyrogens, as well as other impurities that could be harmful to humans or animals.

One will generally desire to employ appropriate salts and buffers to render drugs, proteins or vectors stable and allow for uptake by target cells. Aqueous compositions of the present disclosure comprise an effective amount of the drug, vector or proteins, dissolved or dispersed in a pharmaceutically acceptable carrier or aqueous medium. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Except insofar as any conventional media or agent is incompatible with the active ingredients of the present disclosure, its use in therapeutic compositions is contemplated. Supplementary active ingredients also can be incorporated into the compositions, provided they do not inactivate the vectors or cells of the compositions.

The active compositions of the present disclosure may include classic pharmaceutical preparations. Administration of these compositions according to the present disclosure may be via any common route so long as the target tissue is available via that route. This includes oral, nasal, or buccal. Alternatively, administration may be by intradermal, subcutaneous, intramuscular, intraperitoneal or intravenous injection, or by direct injection into cardiac tissue. Such compositions would normally be administered as pharmaceutically acceptable compositions, as described supra.

The active compounds may also be administered parenterally or intraperitoneally. By way of illustration, solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations generally contain a preservative to prevent the growth of microorganisms.

The pharmaceutical forms suitable for injectable use include, for example, sterile aqueous solutions or dispersions and sterile powders for the extemporaneous preparation of sterile injectable solutions or dispersions. Generally, these preparations are sterile and fluid to the extent that easy injectability exists. Preparations should be stable under the conditions of manufacture and storage and should be preserved against the contaminating action of microorganisms, such as bacteria and fungi. Appropriate solvents or dispersion media may contain, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions may be prepared by incorporating the active compounds in an appropriate amount into a solvent along with any other ingredients (for example as enumerated above) as desired, followed by filtered sterilization. Generally, dispersions are prepared by incorporating the various sterilized active ingredients into a sterile vehicle which contains the basic dispersion medium and the desired other ingredients, e.g., as enumerated above. In the case of sterile powders for the preparation of sterile injectable solutions, the preferred methods of preparation include vacuum-drying and freeze-drying techniques which yield a powder of the active ingredient(s) plus any additional desired ingredient from a previously sterile-filtered solution thereof.

The compositions of the present disclosure generally may be formulated in a neutral or salt form. Pharmaceutically-acceptable salts include, for example, acid addition salts (formed with the free amino groups of the protein) derived from inorganic acids (e.g., hydrochloric or phosphoric acids, or from organic acids (e.g., acetic, oxalic, tartaric, mandelic, and the like. Salts formed with the free carboxyl groups of the protein can also be derived from inorganic bases (e.g., sodium, potassium, ammonium, calcium, or ferric hydroxides) or from organic bases (e.g., isopropylamine, trimethylamine, histidine, procaine and the like.

Upon formulation, solutions are preferably administered in a manner compatible with the dosage formulation and in such amount as is therapeutically effective. The formulations may easily be administered in a variety of dosage forms such as injectable solutions, drug release capsules and the like. For parenteral administration in an aqueous solution, for example, the solution generally is suitably buffered and the liquid diluent first rendered isotonic for example with sufficient saline or glucose. Such aqueous solutions may be used, for example, for intravenous, intramuscular, subcutaneous and intraperitoneal administration. Preferably, sterile aqueous media are employed as is known to those of skill in the art, particularly in light of the present disclosure. By way of illustration, a single dose may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics Standards.

### V. COMBINATION THERAPIES

In another embodiment, it is envisioned to use the mitochondrial transfer vectors of the present disclosure in combination with other therapeutic modalities, such as those discussed above, e.g., LHON. Combinations may be achieved by treating patients with a single composition or pharmacological formulation that includes both agents, or by treating the patient with two distinct compositions or formulations, at the same time, wherein one composition includes the mitochondrial transfer vector and the other includes the agent. Alternatively, the mitochondrial transfer vector therapy may precede or follow administration of the other agent(s) by intervals ranging from minutes to weeks. In embodiments where the other agent and the mitochondrial transfer vector therapy are applied separately to the patient, one would generally ensure that a significant period of time did not expire between the time of each delivery, such that the other agent and the mitochondrial transfer vector therapy would still be able to exert an advantageously combined effect on the cell. In such instances, it is contemplated that one would typically contact the cell with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either the mitochondrial transfer vector therapy or the other agent will be desired. In this regard, various combinations may be employed. By way of illustration, where the mitochondrial transfer vector therapy is “A” and the other agent is “B,” the following permutations based on 3 and 4 total administrations are exemplary:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are likewise contemplated.

### VI. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Methods

Engineering gene therapy constructs to effectively target RNA sequences into the mitochondria. ND6 mtDNA sequences with the recoded mtDNA genetic code are introduced into a variant of the original Koehler and Teitell (UCLA) plasmid pQCXIP which contains the RPLysA construct. These new ND6 constructs were engineered in the pQCXIN retroviral vector background that was purchased from Clontech (Cat# 631514). pQCXIN vector contains neomycin resistance gene which allows selection of positive clones. Chicken beta actin promoter (CAG) was introduced right next to CMV enhancer to enable transcription of the inserted gene cassette. At 3′ end of the CAG promoter Agel restriction site was used to insert the mouse/human 5S (NCBI Reference Sequence: NR_023363.1) sequence functioning as the mitochondrial targeting component of the newly engineered plasmid. Mfe I restriction site was used to integrate a 33 bp pre-tRNA^{Glu} and 69 bp mitochondrial DNA tRNA^{Glu} (TRNE) sequences. Mitochondrial recoded ND6 gene was then inserted after TRNE sequence. This recoded ND6 gene is recoded to ensure that it is only translated in the mitochondrial matrix and not in the cytosol (as explained earlier). One of the key challenges of releasing an unmodified version of the recoded ND6 gene was overcome by introduction of the interface between the tRNA^{Glu} sequence and the inserted ND6 gene. This sequence is identical to that found between genes in the mtDNA so that the imported polycistronic RNA will be properly processed naturally to yield mature translatable ND6 mRNA. 5′ of the pre-tRNA will be processed by the mitochondrial RNase P and the 3′ junction between tRNA^{Glu} and recoded mtND6 will be processed by ELAC2 protein. The inventors engineered the natural tRNA^{Glu} sequence to exploit both RNase P and ELAC2 site-specific phosphodiester hydrolysis activities to process and release the allotopically expressed recoded mtND6 transcript within the mitochondrial matrix. This strategy is extremely important and unique because the mitochondrial translation machinery is not used to seeing modified 5′ ends in an mRNA. If the mitochondrial targeting sequence (5S sequence in this case) was left intact, the transcript would get imported into the mitochondria, however, the possibility of it being recognized as a translatable RNA by the endogenous mitochondrial translational machinery would be extremely rare. Therefore, exploiting the natural machinery of RNA processing is a great idea without having to introduce additional foreign RNA processing molecules thus avoiding unnecessary toxicity. BamH I and EcoR I sites within which a 3X-Flag tag is inserted flank the 3′-end of the recoded ND6 cassette. While only 5 to 10% if the transcribed RNA may enter the mitochondrion, the non-imported mRNA cannot be translated on the cytosolic ribosomes due to the presence in the mtDNA genes of UUG codons which are read as tryptophan in the mitochondrion but as stop codons in the cytosol.

Cell cybrids and generation of stable cell lines. Transient transfection was carried out using Liofectamine LTX reagent (Life Tech) following the manufacture's protocol. To generate stable cell lines, the cell cybrids were selected over 5-6 weeks after transfection using G418 (neomycin analog). The media was changed every 3-4 days with fresh antibiotic. After the neomycin resistant cells started to proliferate, they were transferred to the T25 flask and expanded.

Lactate, glutamate, glucose and glutamine. Media from 0.5×10⁶ cells used to determine lactate, glucose, glutamine and glutamate concentrations using YSI Bioanalyzer. The metabolite production rate was calculated by subtracting the amount of metabolite detected in media without cells from the amount detected in populated wells. The molar amount was then normalized to incubation time and cell number.

High-resolution respiration studies. For high-resolution respirometry in the Oroboros Oxygraph (O2k), the O2k was calibrated to air with cell culture medium (temperature: 37° C., stirrer speed: 750 rpm, gain of the oxygen sensor: 4, data recording interval: 2 sec). Subsequently, the chambers were loaded with the harvested cells, resuspended in their previous, centrifuged (2000 g, 3 min) media at a concentration of 0.8 mio/ml. suspension and chambers were closed with the stoppers, preventing any air exchange. After adding the cells, the first plateau of the oxygen flux resembles the Routine respiration of the cells. Leak respiration is induced by adding 1.25 μM of oligomycin, a complex V inhibitor. Releasing the proton gradient by titrating the uncoupler FCCP (final concentration 1.5 μM to 4.5 μM) results in the electron transport system capacity (ETS capacity). Finally, 1 μM rotenone and 5 mM antimycin A are added, fully inhibiting the electron transport system, resulting in the residual oxygen consumption (ROX). Respiration is displayed as the median oxygen flux per million cells of all the data points of the interval drawn on the plateaus. ROX was subtracted and data were normalized to mtDNA copy number

Reactive oxygen species (ROS) was determined by staining 1 million cells with the cell-permeant chloromethyl derivative of an indicator dye, 2′,7′-dichlorodihydrofluorescein diacetate (H₂DCFDA) (Thermofisher Scientific) following manufacturer's recommended protocol. Each sample was run in triplicate, n=3, in two independent experiments, and results are shown as fold change from WT per condition.

Next Generation sequencing, library preparation and sequencing analysis. Paired-end sequence reads in fastq files were aligned with gsnap version 2018-03-25 (cite: PMID: 20147302) using default alignments to chromosome M of the GRCm38.p3 reference sequence. Duplicate reads were identified using Picard tools version 1.130 (broadinstitute.github.io/picard). Variants were called from the resulting bam files using freebayes, a haplotype-based variant detector version 1.1.0 (Garrison E MG. Haplotype-based variant detection from short-read sequencing. ArXiv Q-Bio. 2012:1207.3907) with arguments “—ploidy 1—min-base-quality 20—no-population-priors—min-alternate-count—hwe-priors-off—min-alternate-count 1—min-mapping-quality 10—use-best-n-alleles 2”.

Electron microscopy imaging and analysis. 2×10⁶ cells for each sample were counted and pelleted for electron microscopic examination after fixing with 2.5% glutaraldehyde, 2.0% paraformaldehyde in 0.1M sodium cacodylate buffer, pH7.4, overnight at 4° C. After subsequent buffer washes, the samples were post-fixed in 2.0% osmium tetroxide for 1 hour at room temperature and rinsed in dH2O prior to en bloc staining with 2% uranyl acetate. After dehydration through a graded ethanol series, the tissue was infiltrated and embedded in EMbed-812 (Electron Microscopy Sciences, Fort Washington, Pa.). Thin sections were stained with uranyl acetate and lead citrate and examined with a JEOL 1010 electron microscope fitted with a Hamamatsu digital camera and AMT Advantage image capture software. The images were analyzed using Fiji software (Schindelin et al., (2012) Nature methods). Each group had >=10 cells. Statistical analysis was performed using Prism 7.0

Mito membrane potential and calcium flux. Control (LM129), ND6 mutant (LMJL2), and ND6 rescue (LMJL2+HsND6) cybrids were washed in Ca²⁺ free PBS, pH 7.4. Equal amounts of cells (7.5×10⁶ cells) were resuspended and permeabilized with 40 μg/ml digitonin in 1.5 ml of intracellular medium (ICM) composed of 120 mM KCl, 10 mM NaCl, 1 mM KH₂PO₄, 20 mM Hepes-Tris, pH 7.2 and 2 μM thapsigargin to block the SERCA pump. All the measurements were performed in the presence of 2 mM succinate to energize the mitochondria. The simultaneous measurement of mitochondrial membrane potential and extramitochondrial Ca²⁺ ([Ca²⁺]ₒᵤₜ) clearance as an indicator of ₘCa²⁺ uptake was achieved by loading the permeabilized cells with JC-1 (800 nM) and Fura2-FF (0.5 μM), respectively. Fluorescence was monitored in a multi-wavelength excitation dual-wavelength emission fluorimeter (Delta RAM, PTI). [Ca²⁺]ₒᵤₜ is represented as the excitation ratio (340 nm/380 nm) of Fura2-FF/FA fluorescence and mitochondrial membrane potential as the ratio of the fluorescence of J-aggregate (570 nm excitation/595 nm emission) and monomer (490 nm excitation/535 nm emission) forms of JC-1. After 20 s of data recording, JC-1 was added. At 350 s an extramitochondrial Ca²⁺ bolus (20 μM) was added to measure the rate of ₘCa²⁺ uptake, followed by addition of 1 μM Ru360 at 550 s to inhibit MCU-mediated Ca²⁺ uptake, 10 μM CGP37157 at 610 s to inhibit NCLX, and 2 μM CCCP at 750 s to dissipate mitochondrial membrane potential. All the experiments were performed at 37° C. with constant stirring (Doonan et al., 2014; Hoffman et al., 2013a; Hoffman et al., 2013b; Mallilankaraman et al., 2012a; Mallilankaraman et al., 2012b).

### Example 2

Production of stable cell line, selection and screening. All cell cybrids were transiently transfected using Lipofectamine 3000. 48 hours post transfection, the cells were grown under G418 selection pressure for the next 6-7 weeks to create stable cell lines. Equal number of cells (0.5×10⁶) were plated in a 6 well plate in quadruplicates. The color of the media was monitored until about day 6-7. The media color changes from pink (alkaline) to yellow (acidic). The cells were trypsinized and counted. mtND6 FS mutant cells showed decreased cell number and yellowing of the media whereas WT cybrids and rescued mutants had higher cell number with alkaline media. The inventors also measured the average diameter and average cellular volume of WT cells and mutant cells. Interestingly, the average diameter and average cellular volume rescue cells did not change and was identical to the mutant cells. This suggests that the allotopic expression does alter the cellular phenotype rather is specific to physiological rescue. There was no difference in viability between the WT and rescued mutant cells.

Cell line characterization by sequencing. The existence of mtND6 frameshift mutation was confirmed by Sanger sequencing by using mtDNA specific primers as well as Next Generation Sequencing (NGS) (SFIG. 1). Homoplasmy levels for each cell lines were confirmed by NGS. LM129 is a wild-type cell cybrid line. LMJL2 cell cybrids has repeatedly shown to contain an insertion of ‘C’ at position 133885 of the mtDNA based on Sanger sequencing (SFIGS. 2A-C). However, the digests for RFLP assay contain a residual uncut band. On confirming with NGS, the inventors found LMJL2 to be homoplasmic mtND6 FS mutant line (SFIGS. 2A-C).

O2k based high-resolution respiration analysis. In high-resolution respirometry in an O2k, mutant cells showed a (significant) 40-50% reduction in Routine and Leak respiration as well as ETS capacity compared to control cells. In the stable rescue cell line, all three respiratory states are (significantly) higher than in the mutant cells, showing a rescue efficiency of 30% (ETS capacity) to 70% (Leak). Interestingly, the respiratory control ratios were altered, neither in mutant nor in the rescued cells.

To exclude that respiratory differences derive from changes in cell size or mitochondrial content, we normalized respiration also to mtDNA copy number for each cell line.

All of the compositions and/or methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and/or methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VII. REFERENCES

The following references, to the extent that they provide exemplary procedural or other details supplementary to those set forth herein, are specifically incorporated herein by reference.
Doonan, P. J., Chandramoorthy, H. C., Hoffman, N. E., Zhang, X., Cardenas, C., Shanmughapriya, S., Rajan, S., Vallem, S., Chen, X., Foskett, J. K., et al. (2014). LETM1-dependent mitochondrial Ca2+ flux modulates cellular bioenergetics and proliferation. FASEB J 28, 4936-4949.
Hoffman, N. E., Chandramoorthy, H. C., Shamugapriya, S., Zhang, X., Rajan, S., Mallilankaraman, K., Gandhirajan, R. K., Vagnozzi, R. J., Ferrer, L. M., Sreekrishnanilayam, K., et al. (2013a). MICU1 motifs define mitochondrial calcium uniporter binding and activity. Cell Rep 5, 1576-1588.
Hoffman, N. E., Chandramoorthy, H. C., Shanmughapriya, S., Zhang, X. Q., Vallem, S., Doonan, P. J., Malliankaraman, K., Guo, S., Rajan, S., Elrod, J. W., et al. (2013b). SLC25A23 augments mitochondrial Ca(2)(+) uptake, interacts with MCU, and induces oxidative stress-mediated cell death. Mol Biol Cell 25, 936-947.
Mallilankaraman, K., Cardenas, C., Doonan, P. J., Chandramoorthy, H. C., Irrinki, K. M., Golenar, T., Csordas, G., Madireddi, P., Yang, J., Muller, M., et al. (2012a). MCUR1 is an essential component of mitochondrial Ca2+ uptake that regulates cellular metabolism. Nat Cell Biol 14, 1336-1343.
Mallilankaraman, K., Doonan, P., Cardenas, C., Chandramoorthy, H. C., Muller, M., Miller, R., Hoffman, N. E., Gandhirajan, R. K., Molgo, J., Birnbaum, M. J., et al. (2012b). MICU1 is an essential gatekeeper for MCU-mediated mitochondrial Ca(2+) uptake that regulates cell survival. Cell 151, 630-644.
Schindelin, J.; Arganda-Carreras, I. & Frise, E. et al. (2012), “Fiji: an open-source platform for biological-image analysis”, Nature methods 9(7): 676-682, PMID 22743772, doi:10.1038/nmeth.2019
Baichwal and Sugden, In: Gene Transfer, Kucherlapati (Ed), NY, Plenum Press, 117-148, 1986.
Benvenisty and Neshif, Proc. Natl. Acad. Sci. USA, 83:9551-9555, 1986.
Chen and Okayama, Mol. Cell Biol., 7:2745-2752, 1987.
Coffin, In: Virology, Fields et al. (Eds.), Raven Press, NY, 1437-1500, 1990.
Coupar et al., Gene, 68:1-10, 1988.
Dubensky et al., Proc. Natl. Acad. Sci. USA, 81:7529-7533, 1984.
Fechheimer et al., Proc Natl. Acad. Sci. USA, 84:8463-8467, 1987.
Ferkol et al., FASEB 1, 7:1081-1091, 1993.
Fraley et al., Proc Natl. Acad. Sci. USA, 76:3348-3352, 1979
Friedmann, Science, 244:1275-1281, 1989.
Ghosh and Bachhawat, In: Liver Diseases, Targeted Diagnosis and Therapy Using Specific Receptors and Ligands, Wu et al. (Eds.), Marcel Dekker, NY, 87-104, 1991.
Ghosh-Choudhury et al., EMBO J., 6:1733-1739, 1987.
Gomez-Foix et al., J. Biol. Chem., 267:25129-25134, 1992.
Gopal, Mol. Cell Biol., 5:1188-1190, 1985.
Graham and Prevec, In: Methods in Molecular Biology: Gene Transfer and Expression Protocol, Murray (Ed.), Humana Press, Clifton, N.J., 7:109-128, 1991.
Graham and van der Eb, Virology, 52:456-467, 1973.
Graham et al., J. Gen. Virol., 36:59-72, 1977.
Grunhaus and Horwitz, Seminar in Virology, 3:237-252, 1992.
Harland and Weintraub, J. Cell Biol., 101:1094-1099, 1985.
Hermonat and Muzycska, Proc. Nat'l Acad. Sci. USA, 81:6466-6470, 1984.
Hersdorffer et al., DNA Cell Biol., 9:713-723, 1990.
Herz and Gerard, Proc. Nat'l. Acad. Sci. USA 90:2812-2816, 1993.
Horwich et al., J. Virol., 64:642-650, 1990.
Jones and Shenk, Cell, 13:181-188, 1978.
Kaneda et al., Science, 243:375-378, 1989.
Kato et al., J Biol Chem., 266(6):3361-3364, 1991.
Klein et al., Nature, 327:70-73, 1987.
Le Gal La Salle et al., Science, 259:988-990, 1993.
Levrero et al., Gene, 101:195-202, 1991.
Mann et al., Cell, 33:153-159, 1983.
Markowitz et al., J. Virol., 62:1120-1124, 1988.
Nicolas and Rubinstein, In: Vectors: A survey of molecular cloning vectors and their uses, Rodriguez and Denhardt (Eds.), Stoneham: Butterworth, 494-513, 1988.
Nicolau and Sene, Biochim. Biophys. Acta, 721:185-190, 1982.
Nicolau et al., Methods Enzymol., 149:157-176, 1987.
Paskind et al., Virology, 67:242-248, 1975.
Perales et al., Proc. Natl. Acad. Sci. USA, 91(9):4086-4090, 1994.
Perales-Clemente et al., Nucleic Acids Res. 39(1):225-3, 2011.
Potter et al., Proc. Natl. Acad. Sci. USA, 81:7161-7165, 1984.
Ragot et al., Nature, 361:647-650, 1993.
Remington's Pharmaceutical Sciences, 15^{th} ed., 1035-1038 and 1570-1580, Mack Publishing Company, P A, 1980.
Renan, Radiother. Oncol., 19:197-218, 1990.
Rich et al., Hum. Gene Ther., 4:461-476, 1993.
Ridgeway, In: Vectors: A survey of molecular cloning vectors and their uses, Stoneham: Butterworth, 467-492, 1988.
Rippe et al., Mol. Cell Biol., 10:689-695, 1990.
Rosenfeld et al., Cell, 68:143-155,1992.
Rosenfeld et al., Science, 252:431-434,1991.
Roux et al., Proc. Natl. Acad. Sci. USA, 86:9079-9083, 1989.
Stratford-Perricaudet and Perricaudet, In: Human Gene Transfer, Cohen-Haguenauer and Boiron (Eds.), John Libbey Eurotext, France, 51-61, 1991.
Stratford-Perricaudet et al., Hum. Gene. Ther., 1:241-256, 1990.
Temin, In: Gene Transfer, Kucherlapati (Ed.), NY, Plenum Press, 149-188, 1986.
Tur-Kaspa et al., Mol. Cell Biol., 6:716-718, 1986.
Varmus et al., Cell, 25:23-36, 1981.
Wagner et al., Proc. Natl. Acad. Sci. USA, 87(9):3410-3414, 1990.
Wong et al., Gene, 10:87-94, 1980.
Wu and Wu, Adv. Drug Delivery Rev., 12:159-167, 1993.
Wu and Wu, Biochemistry, 27:887-892, 1988.
Wu and Wu, J. Biol. Chem., 262:4429-4432, 1987.
Yang et al., Proc. Natl. Acad. Sci. USA, 87:9568-9572, 1990.
Zelenin et al., FEBS Lett, 280:94-96, 1991.

1. An RNA comprising, in a 5′ to 3′ order, a mitochondrial tRNA element, a first open reading frame, and a translation termination signal.

2. The RNA of claim 1, further comprising a mitochondrial signal sequence (mtRSS) located 5′ to said mitochondrial tRNA element.

3. The RNA of claim 1, further comprising a second open reading from downstream of said first open reading frame, and upstream of said translation termination signal.

4. The RNA of claim 1, further comprising a 3′ untranslated region (UTR).

5. The RNA of claim 1, wherein said mitochondrial tRNA element is a TRNE element, such as SEQ ID NO: 1.

6. The RNA of claim 2, wherein said mtRSS is a 5S mtRSS.

7. The RNA of claim 1, wherein the first open reading frame encodes a mitochondrial protein.

8. The RNA of claim 3, wherein the second open reading frame encodes a detectable marker.

9. The RNA of claim 8, wherein the detectable marker is a protein tag (e.g., FLAG) or a fluorescent protein.

10. The RNA of claim 1, wherein the translation termination signal is TAA.

11. An expression cassette comprising, in a 5′ to 3′ order, an RNA polymerase II promoter, a mitochondrial tRNA RNE element, a first open reading frame, and a translation termination signal.

12. The expression cassette of claim 11, further comprising a mitochondrial signal sequence (mtRSS) located 3′ to said RNA polymerase II promoter and 5′ to said mitochondrial tRNA element.

13. The expression cassette of claim 11, wherein said expression cassette further comprises a second open reading from downstream of said first open reading frame, and upstream of said translation termination signal.

14. The expression cassette of claim 11, wherein said expression cassette further comprises a 3′ untranslated region (UTR).

15. The expression cassette of claim 11, wherein said mitochondrial tRNA element is a TRNE element, such as SEQ ID NO: 1.

16. The expression cassette of claim 12, wherein said mtRSS is a 5S mtRSS.

17. The expression cassette of claim 11, wherein the first open reading frame encodes a mitochondrial protein.

18. The expression cassette of claim 13, wherein the second open reading frame encodes a detectable marker.

19. The expression cassette of claim 18, wherein the detectable marker is a protein tag (e.g., FLAG) or a fluorescent protein.

20. The expression cassette of claim 11, wherein the translation termination signal is TAA.

21. The expression cassette of claim 11, wherein the RNA polymerase II promoter (RNAPII) is a eukaryotic RNAPII promoter, such as a chicken β actin promoter or a cytomegalovirus promoter.

22. The expression cassette of claim 11, wherein said expression cassette is comprised in a selectable and/or replicable vector.

23. The expression cassette of claim 22, wherein said replicable vectors is a viral vector, such as a lentiviral vector, or an adeno-associated viral vector.

24. A host cell comprising the RNA of claim 1.

25. A host cell comprising the expression cassette of claim 11.

26. A method of expressing an RNA in a cell comprising contacting a cell with an expression cassette according to claim 11 and culturing said cell under conditions supporting transcription and translation of an RNA encoded by said expression cassette.

27. A method of expressing an RNA in a cell comprising culturing said a host cell according to claim 25 under conditions supporting transcription and translation of an RNA encoded by said expression cassette.

28. A method of complementing a defect in a mutated mitochondrial protein in a cell comprising contacting said cell with an expression cassette comprising, in a 5′ to 3′ order, an RNA Pol II promoter, a mitochondrial tRNA element, a first open reading frame encoding a non-mutant form of said mutated mitochondrial protein, and a translation termination signal.

29. The method of claim 28, further comprising a mitochondrial signal sequence (mtRSS) located 3′ to said RNA polymerase II promoter and 5′ to said mitochondrial tRNA element.

30. The method of claim 28, wherein said expression cassette further comprises a second open reading from downstream of said first open reading frame, and upstream of said translation termination signal.

31. The method of claim 28, wherein said expression cassette further comprises a 3′ untranslated region (UTR).

32. The method of claim 28, wherein said tRNA element element comprises a TRNE element, such as SEQ ID NO: 1.

33. The method of claim 29, wherein said mtRSS is a 5S mtRSS.

34. The method of claim 30, wherein the second open reading frame encodes a detectable marker.

35. The method of claim 34, wherein the detectable marker is a protein tag (e.g., FLAG) or a fluorescent protein.

36. The method of claim 28, wherein the translation termination signal is TAA.

37. The method of claim 28, wherein the RNA polymerase II promoter (RNAPII) is a eukaryotic RNAPII promoter, such as a chicken β actin promoter or a cytomegalovirus promoter.

38. The method of claim 28, wherein said expression cassette is comprised in a selectable and/or replicable vector.

39. The method of claim 38, wherein said selectable and/or replicable vectors is a viral vector.

40. The method of claim 39, wherein said viral vector is a retroviral vector, a lentiviral vector or an adeno-associated viral vector.

41. A method of complementing a defect in a mutated mitochondrial ND6 protein in a cell comprising contacting said cell with a expression cassette comprising, in a 5′ to 3′ order, an RNA Pol II promoter, a mitochondrial tRNA element, a first open reading frame encoding a non-mutant form of said mutated ND6 protein, and a translation termination signal.

42. The method of claim 41, further comprising a mitochondrial signal sequence (mtRSS) located 3′ to said RNA pol II promoter and 5′ to said tRNA element.

43. The method of claim 41, wherein said expression cassette further comprises a 3′ untranslated region (3′ UTR).

44. The method of claim 41, wherein said tRNA element is a TRNE element, such as SEQ ID NO: 1.

45. The method of claim 41, wherein said mtRSS is a 5S mtRSS.

46. The method of claim 41, wherein the translation termination signal is TAA.

47. The method of claim 41, wherein the RNA polymerase II (RNAPII) promoter is a eukaryotic or RNAPII promoter, such as a chicken β actin promoter or a cytomegalovirus promoter.

48. The method of claim 41, wherein said expression cassette is comprised in a replicable vector.

49. The method of claim 48, wherein said replicable vectors is a viral vector.

50. The method of claim 49, wherein said viral vector is a retroviral vector, lentiviral vector or an adeno-associated viral vector.

51. The method of claim 41, wherein said cell is located in a subject, and said expression cassette is administered to said subject.

52. The method of claim 51, wherein said subject has been diagnosed with a primary mitochondrial disease, such as Leber's Hereditary Optic Neuropathy (LHON).

53. The method of claim 52, further comprising administering a second LHON therapy to said subject.

54. The method of claim 51, further comprising administering said expression cassette at least a second time, such as chronically.

55. The method of claim 51, wherein said subject is a human or non-human animal.
